(19) United States
(12) Patent Application Publication
(10) Pub. No.: US 2024/0307194 A1
(43) Pub. Date: Sep. 19, 2024

(54) SYSTEM AND METHODS FOR GENERATING TOUCH SIGNAL CORRESPONDING TO STATE OF SUBJECT

(71) Applicant: KING FAHD UNIVERSITY OF PETROLEUM AND MINERALS, Dhahran (SA)

(72) Inventors: Asad Muhammad BUTT, Dhahran (SA); Khurram Karim QURESHI, Dhahran (SA); Hassan ALSAFFAR, Dhahran (SA); Muhannad ALSHAREEF, Dhahran (SA); Rayan AWARI, Dhahran (SA)

(73) Assignee: KING FAHD UNIVERSITY OF PETROLEUM AND MINERALS, Dhahran (SA)

(21) Appl. No.: 18/184,106

(22) Filed: Mar. 15, 2023

(51) Int. Cl. A61F 2/72; G06F 3/01

(52) U.S. Cl. CPC A61F 2/72; G06F 3/015; A61F 2002/5061; A61F 2002/6827; A61F 2002/6881; A61F 2002/704; A61F 2002/705; G06F 2203/011

(57) ABSTRACT

A method of generating a touch signal corresponding to a state of a subject, the method including, receiving, from a foot of the subject, multiple wavelength signals corresponding to an applied plantar pressure based on the state of the subject. The method further includes receiving, using channels of a BCI mounted on the subject's head, a plurality of EEG signals (brain signals) that corresponds with wavelength signals. The method further includes transmitting EEG signals to train a classifier to identify a correlation between EEG signals and wavelength signals. The method further includes selecting a subsection of channels with high correlation with wavelength signals. The method further includes forming a secondary dataset by combining the subsection of channels with wavelength signals. The secondary dataset is passed to train a ML model to generate the touch signal corresponding to the subject's state that is relayed to a lower limb prosthesis.

### STATEMENT REGARDING PRIOR DISCLOSURES BY THE INVENTOR(S)

Aspects of this technology are described in Butt, Asad Muhammad, et al. “AI Prediction of Brain Signals for Human Gait Using BCI Device and FBG Based Sensorial Platform for Plantar Pressure Measurements.” Sensors, vol. 22, no. 8, April 2022, p. 3085, doi.org/10.3390/s22083085.

### STATEMENT OF ACKNOWLEDGEMENT

This research was supported by King Fahd University of Petroleum and Minerals under the project number SR191027.

### BACKGROUND

### Technical Field

The present disclosure is directed towards generating touch sensation in prosthetic devices, and more particularly relates to a system and methods for generating a touch signal corresponding to a state of a subject.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Human gait and other clinical investigations related to human biomechanics are useful in analyzing a root cause for patients suffering from impediments in locomotion. The impediments may arise due to numerous circumstances such as injuries, neurological disorders and adaptation to prosthetic devices in case of amputation. FIG. 1 illustrates a known human gait representation 100. While walking, successive actions are performed by the human for maintaining contact of the foot with the ground and maintaining balance of the body, are shown in FIG. 1. Walking includes plurality of steps such as an initial contact step 104 of a left foot 102, for example, on the ground, followed by a loading response step 106. In the loading response step 106, weight of body begins to apply a gradually increasing pressure only on the left foot. A mid-stance step 108 occurs followed by the loading response step 106 where a complete weight of the body comes over the left foot. A terminal stance step 110 occurs followed by the mid-stance step 108 where the right foot overtakes the left foot in response to the further movement of the body. A pre-swing step 112 occurs followed by the terminal stance step 110 when the left foot 102 is about to untouch the ground. An initial swing step 114 occurs followed by the pre-swing step 112 when the left foot 102 has untouched the ground and the whole body weight begin to gradually apply pressure on the right foot 120. A midswing step 116 occurs followed by the initial swing step 114 when the weight of the body is only on the right foot 120 and the left foot 102 is about to again touch the ground. A terminal swing step 118 occurs followed by the midswing step 116 where the left foot 102 has again touched the ground, and the body weight begins to apply gradual pressure on the left foot again. During motion of a subject, these all steps are repeated continuously. Similarly, other human postures such as sitting and standing are also established using foot contact with the ground. In each of these postures or state, perception of these postures in a brain through pressure profiles or plantar pressure on the sole is developed during various contact scenarios that also enables maintaining a stable gait. The human foot sole has numerous plantar nerves that carry information to a brain motor cortex, where it is perceived as the foot being touched to the ground and interpreted for an adequate response such as to continue making balance while walking, for example.

In a healthy person, brain electroencephalography (EEG) provides a valuable insight into the patient's perception of touch such as a touch of the foot to the ground. When a subject loses a leg or hand due to injury or an accident, i.e., amputation, the sensory loss disables the patient's perception of foot or hand contact with the ground or any surface. Amajor reason for the occurance of amputation is diabetes which is a major concern in the Middle East and North Africa (MENA) region, such as Saudi Arabia. As such, people suffering from amputation have to wear prosthetic devices so that they can perform their daily tasks such as walking or standing like a normal person. Wearing prosthetic devices for amputation is well known in the art. However, the touch sensation or the touch signal is no longer perceived when the prosthetic device touches the ground or any surface at the time of setting, standing and walking. It as been found in research that in case of sensory loss, the brain motor cortex retains the memory of the pre-amputation sensorial feedback that could enable patients to regain sensory feelings. However, there is no prosthetic device known that permits an amputee to feel the sensation of touch. Therefore, there exists a need for a system and/or method that functions to permit an amputee to have a sensation of touch through a prosthetic device attached to the amputee's body.

### SUMMARY

In an exemplary embodiment, the present disclosure discloses a method of generating a touch signal corresponding to a state of a subject. The method includes receiving, with a processing circuitry of a computer controller, a plurality of wavelength signals corresponding to an applied plantar pressure from a foot of the subject, the applied plantar pressure from the foot of the subject corresponding with the state of the subject. The method further includes receiving, with the processing circuitry of the computer controller, a plurality of electroencephalography (EEG) signals corresponding to brain signals of the subject. Each signal of the plurality of EEG signals corresponds with one signal of the plurality of wavelength signals. Each signal of the plurality of EEG signals is registered by one channel of a plurality of channels on a brain control interface (BCI) mounted on the subject's head. The method further includes transmitting, via the plurality of channels, the plurality of EEG signals to a classifier. The method further includes training, with the processing circuitry of the computer controller, the classifier using the plurality of EEG signals, the classifier identifying a correlation between the plurality of EEG signals and the plurality of wavelength signals. The method further includes selecting, via the classifier, a subsection of channels from the plurality of channels with a high correlation to the plurality of wavelength signals. The method further includes combining, with the processing circuitry of the computer controller, the subsection of channels with the plurality of wavelength signals to form a secondary dataset, the secondary dataset being passed to a machine learning model. The method further includes training, with the processing circuitry of the computer controller, the machine learning model using the secondary dataset to generate the touch signal corresponding to the subject's state. The touch signal is relayed to a lower limb prosthesis. The touch signal elicits a subject movement response. The subject movement response comprises a movement of a foot of the lower limb prosthesis and the movement of the foot of the lower limb prosthesis corresponds to the subject's state.

In another exemplary embodiment, the touch signal is transmitted from the lower limb prosthesis to a haptic feedback system. The haptic feedback system comprises a vest worn on the subject's chest. The touch signal elicits a haptic response corresponding to the subject's state.

In another exemplary embodiment, the plurality of channels comprises 16 channels. The subsection of channels selected by the classifier comprises 6 channels.

In another exemplary embodiment, each of the 16 channels comprises an electrode affixed to a crown of the subject's head.

In another exemplary embodiment, each of the plurality of wavelength signals comprises a unique wavelength signal.

In another exemplary embodiment, the foot of the subject is segmented into eight distinct regions. Each of a plurality of sensors on the foot of the subject is fixed to at least one of the eight distinct regions. Each of the plurality of sensors on the foot of the subject cannot be fixed to the same distinct region.

In another exemplary embodiment, the subject's state comprises a sitting position, a standing position, and a walking movement.

In another exemplary embodiment, a walking gait analysis apparatus is disclosed. The walking gait analysis apparatus includes a computer controller, a brain control interface (BCI) and a plurality of sensors. The plurality of sensors are disposed on a toe, a midfoot, and a heel of an insole and configured to output a plurality of wavelength signals. The plurality of wavelength signals corresponds to an applied plantar pressure from a foot of a subject. The plurality of sensors connects to an optical circulator, a light source, and an optical interrogator. The computer controller is configured to receive the plurality of wavelength signals. The computer controller is configured to receive, with processing circuitry, the plurality of wavelength signals corresponding to the applied plantar pressure from the foot of the subject and an electroencephalography (EEG) signal corresponding to the brain signals of the subject. The EEG signal is transmitted to the processing circuitry of the computer controller by the BCI mounted on the subject's head. The plurality of wavelength signals and the brain signals correspond to a state of the subject. The processing circuitry of the computer controller receives a plurality of wavelength signals corresponding to an applied plantar pressure from a foot of the subject. The applied plantar pressure from the foot of the subject corresponds with the state of the subject. The processing circuitry of the computer controller receives a plurality of electroencephalography (EEG) signals corresponding to brain signals of the subject. Each signal of the plurality of EEG signals corresponds with one signal of the plurality of wavelength signals and each signal of the plurality of EEG signals is registered by one channel of a plurality of channels on the BCI mounted on the subject's head. The plurality of channels transmits the plurality of EEG signals to a classifier. The processing circuitry of the computer controller trains the classifier using the plurality of EEG signals. The classifier identifies a correlation between the plurality of EEG signals and the plurality of wavelength signals. The classifier selects a subsection of channels from the plurality of channels with a high correlation to the plurality of wavelength signals. The processing circuitry of the computer controller combines the subsection of channels with the plurality of wavelength signals to form a secondary dataset. The secondary dataset is passed to a machine learning model. The processing circuitry of the computer controller trains the machine learning model using the secondary dataset to generate a walking gait analysis signal corresponding to a subject's state.

In another exemplary embodiment, the state of the subject comprises a sitting position, standing position, or a walking movement.

In another exemplary embodiment, each of the plurality of sensors possess a baseline wavelength signal. A wavelength shift signal is calculated by the optical interrogator based on a difference between the baseline wavelength signal and a peak wavelength signal. Each of the plurality of sensors produces the peak wavelength signal corresponding to the applied plantar pressure from the foot of the subject.

In another exemplary embodiment, the plurality of sensors comprises a first sensor that is fixed to the toe, a second sensor that is fixed to the midfoot and a third sensor that is fixed to the heel.

In another exemplary embodiment, the walking gait apparatus further includes a wearable sandal arrangement, the insole, and a Velcro strap-on. The insole is fixed to the underside of the wearable sandal arrangement and the Velcro strap-on is fixed to the top of the wearable sandal arrangement.

In another exemplary embodiment, The plurality of sensors is coated with a protective layer.

In another exemplary embodiment, a system of plantar pressure response is disclosed in which an applied plantar pressure is registered by a plurality of fiber bragg grating (FBG) sensors. The plurality of FBG sensors are disposed on an insole. A light source illuminates the plurality of FBG sensors. The plurality of FBG sensors each outputs a wavelength shift in response to an applied plantar pressure. An optical circulator provides a three-way gateway between the light source, an optical interrogator, and the plurality of FBG sensors. The optical circulator is connected to the optical interrogator. The wavelength shift travels through the optical circulator from the plurality of FBG sensors to the optical interrogator. The optical interrogator displays the wavelength shift corresponding to each of the plurality of FGB sensors.

In another exemplary embodiment, each of the plurality of FGB sensors possess a base wavelength. The base wavelength of each of the plurality of FGB sensors being a unique wavelength. The wavelength shifts of each of the plurality of FGB sensors do not overlap. A wavelength signal is calculated, via processing circuitry of the optical interrogator, as the difference between the base wavelength and the wavelength shift.

In another exemplary embodiment, each of the plurality of FGB sensors outputs a unique wavelength shift in response to an applied plantar pressure.

In another exemplary embodiment, the interrogation monitor includes a display unit. The wavelength shift from each of the plurality of FGB is projected on the display unit.

In another exemplary embodiment, a region ranging from 10 nanometers to 20 nanometers along a fiber length of the FBG is etched by ultraviolet (UV) radiation.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates an exemplary human gait representation known in the art.

FIG. 2A illustrates a walking gait analysis apparatus, according to certain embodiments.

FIG. 2B illustrates an exemplary illustration of a brain control interface (BCI), according to certain embodiments.

FIG. 2C illustrates a 16-channel based BCI, according to certain embodiments.

FIG. 2D illustrates an exemplary model of the 16 channels-based BCI, according to certain embodiments.

FIG. 3 illustrates brain activity pattern in response to foot, hand and lip movements, according to certain embodiments.

FIG. 4A illustrates a BCI classification stage, according to certain embodiments.

FIG. 4B illustrates a flow diagram of data classification in the computer controller, according to certain embodiments.

FIG. 5A illustrates a raw electroencephalography (EEG) data of the 16 channels during walking of a subject, according to an embodiment.

FIG. 5B illustrates a graph as a normalized value of a preprocessed EEG signal dataset, according to certain embodiments.

FIG. 5C illustrates a graph as a detrended value of the preprocessed EEG signal dataset, according to certain embodiments.

FIG. 5D illustrates a graph of the pre-processed EEG signal dataset after passing detrended value with a low pass filter, according to certain embodiments.

FIG. 6A illustrates a 4-level wavelet decomposition pattern of 16 channels, according to certain embodiments.

FIG. 6B illustrates a 4-level wavelet decomposition pattern of 16 channels with removed level 1 and approximation of coefficients, according to certain embodiments.

FIG. 7A-FIG. 7C illustrate a Fourier transform pattern of the raw EEG signal, processed signals using a first method and the processed signal using a second method, respectively, according to certain embodiments.

FIG. 8A illustrates a confusion matrix based on a result of predicted value and actual value of each model, according to certain embodiments.

FIG. 8B illustrates a graphical representation of channels identified as most sensitive to state of the subject, according to certain embodiments.

FIG. 9A illustrates geometry of a foot sole of a normal leg, according to certain embodiments.

FIG. 9B illustrates points of the foot sole identified as critically imperative points for plantar pressure measurement, according to certain embodiments.

FIG. 9C further illustrates foot pressure identified with markings on an ink pad, according to certain embodiments.

FIG. 10A illustrates a walking gate analysis apparatus, according to certain embodiments.

FIG. 10B illustrates a physical setup of the walking gate analysis apparatus, according to certain embodiments.

FIG. 10C illustrates an insole and a sensor setup in the insole, according to certain embodiments.

FIG. 10D illustrates a wearable sandal arrangement, according to certain embodiments.

FIG. 10E shows an exemplary illustration of a subject wearing the wearable sandal arrangement along with the BCI on his head for predicting the brain signals and/or touch signal, according to certain embodiments.

FIG. 11 illustrates a diagram showing a working principle of a fiber bragg grating (FBG) sensor, according to an embodiment.

FIG. 12A illustrates an exemplary graphical user interface of the optical interrogator, according to certain embodiments.

FIG. 12B illustrates an exemplary user interface illustrating a control panel for 16 channel EEG data collection, according to an embodiment.

FIG. 13A illustrates the BCI prediction and a touch signal generation process, according to certain embodiments.

FIG. 13B illustrates a walking state, a standing state and a sitting state of the subject while wearing the wearable sandal arrangement and the BCI, according to certain embodiments.

FIG. 14 illustrates a signal flow diagram for machine learning process for predicting the brain signal and generating the touch signal, according to certain embodiments.

FIG. 15 illustrates a block diagram of each machine learning model, according to certain embodiments.

FIG. 16A illustrates an experimentally computed wavelength shift registered by plurality of FBG sensor in a standing state, according to certain embodiments.

FIG. 16B illustrates an experimentally computed wavelength shift registered by plurality of FBG sensor in a sitting state after normalization, according to certain embodiments.

FIG. 16C illustrates an experimentally computed wavelength shift registered by plurality of FBG sensor in standing state after normalization, according to certain embodiments.

FIG. 16D illustrates an experimentally computed wavelength shift registered by plurality of FBG sensor in walking state after normalization, according to certain embodiments.

FIG. 17A illustrates an exemplary plot of the values of the training and validation mean square error (MSE) of LSTM machine learning model for the processed data on channel 6, according to certain embodiments.

FIG. 17B illustrates an exemplary plot of the values of the training and validation MSE of RNN machine learning model for the processed data on channel 6, according to certain embodiments.

FIG. 17C illustrates an exemplary plot of the values of the training and validation MSE of GRU machine learning model for the processed data on channel 6, according to certain embodiments.

FIG. 17D illustrates an exemplary plot of the values of the training and validation MSE of LSTM machine learning model for wavelet decomposition data on channel 9, according to certain embodiments.

FIG. 17E illustrates an exemplary plot of the values of the training and validation MSE of RNN machine learning model for wavelet decomposition data on channel 9, according to certain embodiments.

FIG. 17F illustrates an exemplary plot of the values of the training and validation MSE of GRU machine learning model for wavelet decomposition data on channel 9, according to certain embodiments.

FIG. 18A illustrates a comparison graph between an original and predicted EEG signal data at channel 2 for an unknown FBG data, according to certain embodiments.

FIG. 18B illustrates a magnified graph of a portion of FIG. 18A from 1.90 seconds to 2.25 seconds, according to certain embodiments.

FIG. 18C illustrates a magnified graph of a portion of FIG. 18A from 3.4 seconds to 3.8 seconds, according to an exemplary embodiment.

FIG. 19 illustrates a flowchart of a method of generating a touch signal corresponding to a state of a subject, according to an embodiment.

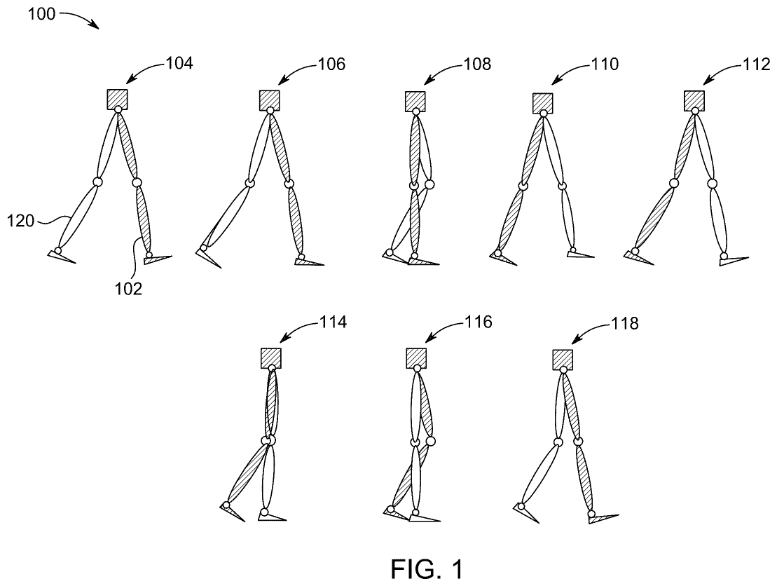

### DETAILED DESCRIPTION

In the drawings, like reference numerals designate identical or corresponding parts throughout the several views. Furthermore, the terms “approximately,” “approximate,” “about,” and similar terms generally refer to ranges that include the identified value within a margin of 20%, 10%, or preferably 5%, and any values therebetween.

Aspects of this disclosure are directed to a system and method of generating a touch signal corresponding to a state of a subject.

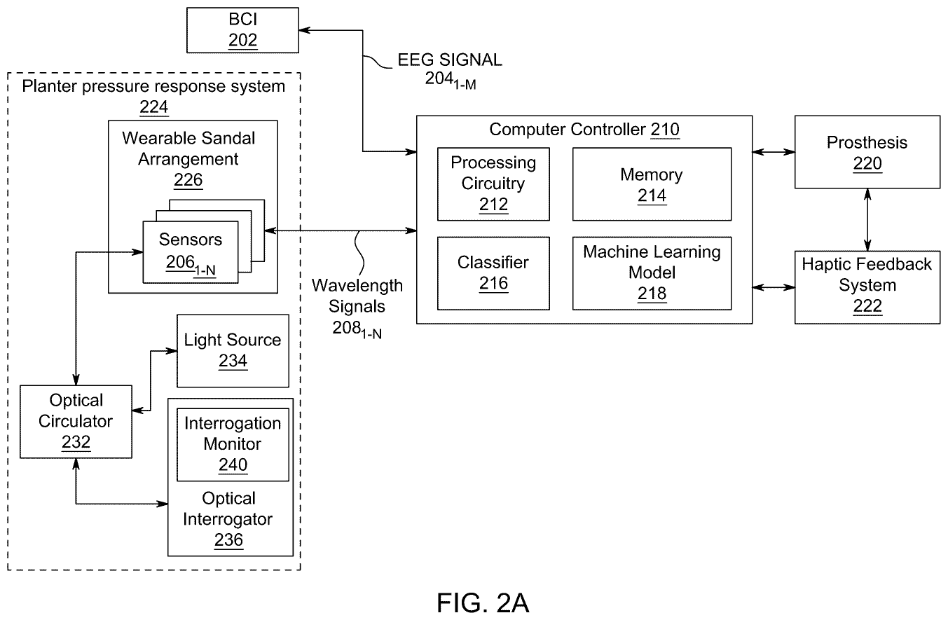

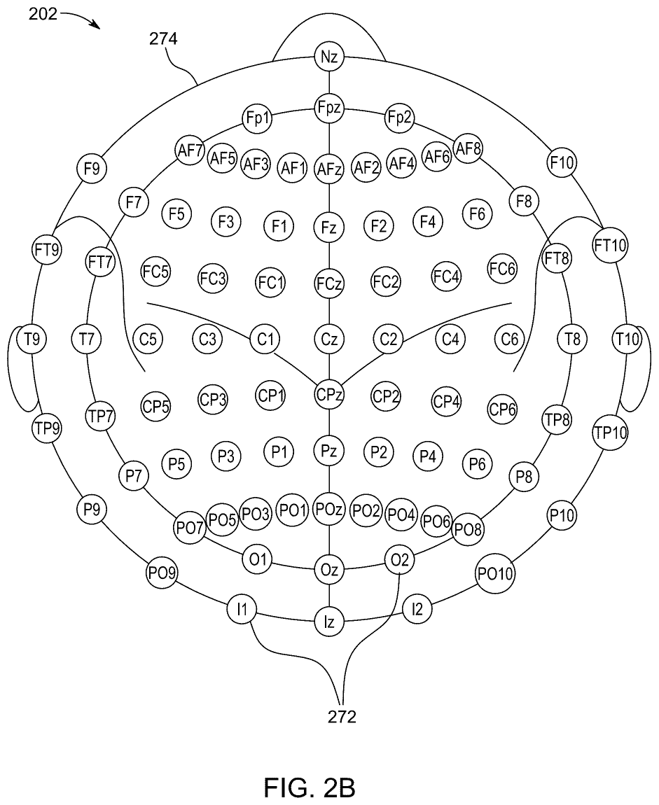

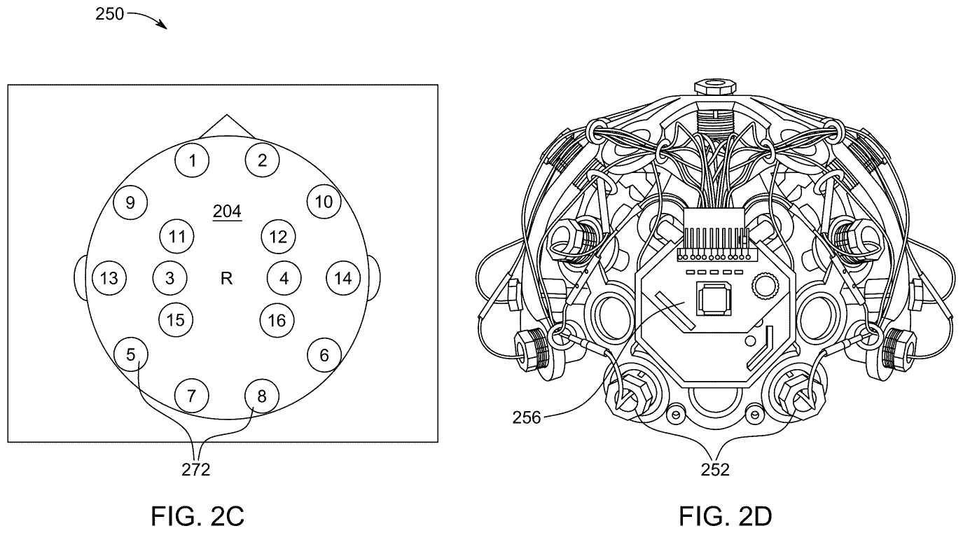

FIG. 2A is a walking gait analysis apparatus 200, according to one or more embodiments. The walking gait analysis apparatus 200 includes, inter alia, a brain control interface (BCI) 202, a plurality of sensors 206_{1-N}, a computer controller 210, processing circuitry 212, a memory 214, a classifier 216, a machine learning model 218, prosthesis 220 and a haptic feedback system 222.

The BCI 202 is a device configured to acquire brain signals and communicate the acquired signals as outputs. An exemplary illustration of the BCI 202 is provided in FIG. 2B, which is a 10-20 BCI 202. The 10-20 system or International 10-20 system is an internationally recognized standard to describe and apply electrodes 272 at defined scalp locations of the head. The “10” and “20” may refer to the distances between adjacent electrodes 272 between 10% to 20% of the total front-back or right-left distance of the head scalp 274. In an example, the BCI 202 is simply a head worn device that includes plurality of electrodes 272. The BCI 202 is mounted on the head such that the plurality of electrodes 272 are in physical contact with the head scalp 274. When the BCI 202 is powered, the 10-20 BCI 202 captures electrical impulses generated by neurons firing in the brain through electrodes 272 and produces brain mapping of various human activities. The captured electrical impulses are referred to as a plurality of electroencephalography (EEG) signals. FIG. 2C illustrates an exemplary 16-channel based BCI 250 having 16 electrodes for capturing the electrical impulses. The 16 electrodes are numbered from 1-16 as shown in the figure. Each electrode is communicatively coupled to corresponding channels of the 16 channels. As described, each of the electrodes is affixed to a crown of the subject's head scalp 274 for obtaining the EEG signals.

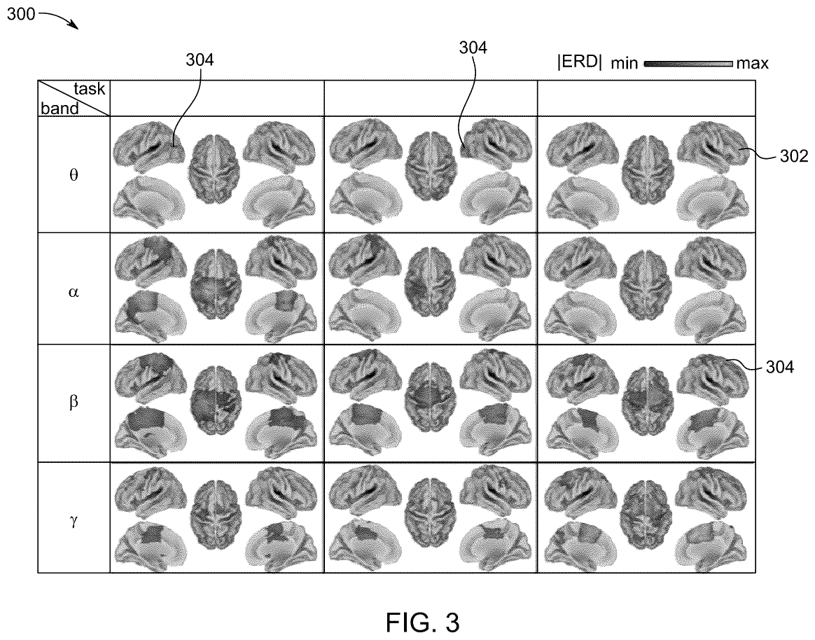

FIG. 2D illustrates an exemplary prototype electrode cap model of the 16 channel based BCI 202, according to an embodiment. As shown, the electrode cap model BCI 202 may be in the form of a structure that can be placed on the head of a subject. The electrode cap model BCI 202 includes plurality of electrodes 272 attached at defined locations to obtain the EEG signals. The BCI 202, as shown in FIG. 2C and FIG. 2D, includes 16 electrodes 272 that provide 16 different channels for identifying the brain signals. The BCI 202 may include a controller 256. Plurality of electrodes 272 are communicatively coupled to the controller 256, either wired or wireless, to capture and provide EEG signals at the time of performing plurality of activities, such as sitting, standing and walking. An example of the BCI 202 is produced by OpenBCI (See: OPENBCI, 67 West St, Brooklyn, New York, United states). FIG. 3 illustrates brain activity pattern 300 against foot, hand and lip movement, according to an embodiment. Whenever, a subject performs a body motion, such as a foot, hand or lips, various parts of a brain 302 generate corresponding brain signals 304. For example if a person moves his or her hand, corresponding brain signals 304 are generated at plurality of locations, as shown in first column of FIG. 3. Similarly, if the subject moves his or her foot, corresponding brain signals 304 are again generated at a plurality of similar or other locations. These brain signals are mapped to corresponding portions of the brain and are shown in second and third column, respectively, of FIG. 3. The disclosure identifies a number and/or position of electrodes of the BCI 202 that captures a maximum response of the brain signals corresponding to the body motion or the subject's state, such as sitting, standing or walking state.

Referring back to FIG. 2A, the plurality of sensors 206_{1-N} include sensors that are disposed over various parts of the body such as a toe, a midfoot, a heel of an insole, palms, etc. For example, a first sensor 206₁ may be fixed to the toe, a second sensor 206₂ may be fixed to the midfoot, a third sensor 206₁ may be fixed to the heel, etc. In some example implementations, the plurality of sensors_{1-N} may be implemented in a wearable sandal arrangement 226 having an insole and Velcro strap-on, where the insole is fixed to the underside of the wearable sandal arrangement and the Velcro (e.g., releasable) strap-on is fixed to the top of the wearable sandal arrangement. In an example, the second sensor 206₂ and the third sensor 206₁ may be placed to contact the midfoot and the heel, respectively. The sensors 206_{1-N} placed in the wearable sandal arrangement are coated with a protective layer such as using rubber, plastics and the like, to prevent any damages. In some examples, the plurality of sensors 206 may be fiber bragg grating (FBG) sensors. The FBG sensors include distributed Bragg reflectors (hereinafter interchangeably referred to as sensors 206_{1-N}) in a short segment of optical fiber that reflects particular wavelength light and transmits all others. Each of the plurality of sensors 206_{1-N} possess a baseline wavelength signal. In some examples, the base wavelength of each of the plurality of FGB sensors 206_{1-M} may be a unique wavelength.

The plurality of sensors 206 are coupled to an optical circulator 232, a light source 234, and an optical interrogator 236. The optical circulator 232 is an optical device having at least three ports configured to direct any light entering one port to exit from another port. In the disclosure, the optical circulator 232 is coupled to the light source 234. The light source 234 is a broadband light source having wavelength in a range of 1510-1590 nm. The light source 234 is used to illuminate the plurality of sensors 206₁-M through the optical circulator 232. The optical interrogator 236 is an optoelectronic instrument configured to read the sensors 206_{1-N}. In the current disclosure, the optical circulator 232 is configured to direct the light from the light source 234 towards the sensors 206_{1-N}, and the light from the sensors 206_{1-N} to the optical interrogator 236. When attached to the various parts of body, the sensors 206_{1-N} output a light with a corresponding plurality of wavelength signals 208_{1-N} in response to applied plantar pressure. In some examples, each of the plurality of sensors 206_{1-N} outputs a unique wavelength shift in response to the applied plantar pressure. The unique wavelength shifts are such that the wavelength shifts of each of the plurality of sensors 206_{1-N} do not overlap.

Based on the light obtained from one or more sensor 206_{1-N}, the optical interrogator 236 is configured to calculate a wavelength shift signal of the corresponding sensor 206_{1-N} based on a difference between the baseline wavelength signal and a peak wavelength signal. The optical interrogator 236 calculates a wavelength signal as the difference between the base wavelength and the wavelength shift.

In an aspect, the each of the plurality of sensors 206_{1-N} produces the peak wavelength signal corresponding to the applied plantar pressure from the foot of the subject. The optical interrogator 236 may include an interrogating monitor 240. The interrogating monitor 240 provides the wavelengths and/or wavelength shifts of each of the sensors 206_{1-N}. The interrogating monitor 240 may include a display device or may be connected to an external display device through which the interrogating monitor 240 displays the wavelengths or wavelength shifts corresponding to each of the plurality of sensors 206_{1-N}. In an example, the interrogating monitor 240 projects the wavelength shift from each of the plurality of sensor 206_{1-N}.

In one or more embodiments, the wearable sandal arrangement 226 (including the plurality of sensor 206_{1-N}), the optical circulator 232, the light source 234, the optical interrogator 236 (having the interrogating monitor 240) may form a plantar-plantar pressure response system 224. The plantar-plantar pressure response system 224 or the sensors 206_{1-N} may communicate the wavelength signals 208_{1-N} to the computer controller 210.

The computer controller 210 is configured to process the EEG signals 204_{1-M} and the wavelength signals 208_{1-N} to generate to generate the touch signal corresponding to the subject's state and/or a walking gait analysis signal corresponding to a subject's state. The computer controller 210 may refer to any computing device such as a computer, a laptop, a desktop, a cloud server or the like. In an embodiment, the computer controller 210 may be a wearable computer. The computer controller 210 includes a processing circuitry 212, a memory 214, a classifier 216, and a machine learning model 218. In an aspect, the BCI 202 is connected either wired or wirelessly with the computer controller 210. The processing circuitry 212 may include hardware circuits that process data from external devices such as the BCI 202 or the plurality of sensors 206_{1-N}. The memory 214 supports the computer controller 210 in various operations including storing data, intermediate data, and processed data.

The classifier 216 may refer to a type of machine learning code that uses rules to assign a class label to a data inputs. In the current disclosure, the classifier 216 is configured to identify a correlation between the plurality of EEG signals and the plurality of wavelength signals (explained in greater detail below). The machine learning model 218 is a code configured to, for example, recognize patterns or behaviors from a dataset based on previous data. In the current disclosure, the machine learning model 218 is configured to generate the touch signal corresponding to the subject's state and/or a walking gait analysis signal corresponding to a subject's state. The touch signal is preferably communicated to the prosthesis 220.

The prosthesis 220 is a device designed to replace a missing part of the body or to augment the existing part of the body to improve functionality. Examples of the prosthesis include transradial, transfemoral, transtibial, transhumeral and other prosthesis. In explanations provided in the current disclosure, the prosthesis is preferably associated with a lower limb, although the system and method can be equally applied to any prosthesis for the body. The prosthesis 220 may communicate the touch signal to the haptic feedback system 222. The haptic feedback system 222 is a device that is configured to create an experience of touch by applying forces, vibrations, or motions to the user.

In operation, a subject is prepared for gait analysis. The preparation includes attaching the walking gait analysis apparatus 200 to the subject. Attaching the walking gait analysis apparatus 200 to the subject includes mounting the BCI 202 device on the head of the subject such that electrodes are in contact with the scalp of the subject, and coupling the plurality of sensors 206_{1-N} to a toe, a midfoot, a heel of an insole, etc.

According to the disclosure, the foot of the subject is segmented into eight distinct regions, and each of the plurality of sensors 206_{1-N} on the foot of the subject is fixed to at least one of the eight distinct regions. Also, each of the plurality of sensors on the foot of the subject is not fixed to the same distinct region.

The BCI 202 and the sensors 206_{1-N} are coupled to the computer controller 210. The computer controller 210 is connected to prosthesis 220 and haptic feedback system 222. In an example, the BCI 202 may be 16-channel BCI that includes 16 channels coupled to corresponding electrodes. The electrodes are affixed to a crown of the subject's head. The subject is made to go through various states including a sitting state, a standing state and a walking state to obtain subject data from the BCI 202 and the sensors 206_{1-N}. These states may cause different applied plantar pressure obtained from a foot of the subject. As a result of different applied plantar pressure, the sensors 206_{1-N} may generate the plurality of wavelength signals 208_{1-N} corresponding to the different states of the subject.

Referring back to FIG. 2A, the processing circuitry 212 of the computer controller 210 receives the plurality of wavelength signals 208_{1-N} corresponding to the applied plantar pressure from the foot of the subject. In aspects, each of the plurality of wavelength signals 208_{1-N} comprises a unique wavelength signal. The processing circuitry 212 of the computer controller 210 also receives the plurality of EEG signals 204_{1-M} corresponding to the brain signals of the subject from the BCI 202. Each signal of the plurality of EEG signals 204_{1-M} corresponds with one signal of the plurality of wavelength signals, and each signal of the plurality of EEG signals 204_{1-M} is registered by one channel of a plurality of channels on the BCI 202 mounted on the subject's head. The computer controller 210 communicates via the plurality of channels, the plurality of EEG signals 204_{1-M} to the classifier 216. The classifier 216 is trained using the plurality of EEG signals 204_{1-M} and the plurality of wavelength signals 208_{1-N}.

Based on the training, the classifier 216 identifies a correlation between the plurality of EEG signals 204_{1-M} and the plurality of wavelength signals 208_{1-N}. In some embodiments, the classifier 216 selects a subsection of channels from the plurality of channels with a high correlation to the plurality of wavelength signals 208_{1-N}. In an example, the classifier selects the subsection of channels based on identifying the number and/or position of electrodes of the BCI 202 that is most responsive in detecting activities of the body. In some examples, if the plurality of channels include 16 channels, the subsection of channels selected by the classifier 216 may include 6 channels. In some other examples, the classifier 216 may select lesser than or more than 6 channels.

The computer controller 210 combines the subsection of channels with the plurality of wavelength signals to form a secondary dataset. The secondary dataset is passed to the machine learning model 218. In some embodiments, the machine learning model 218 is trained using the secondary dataset to generate the touch signal corresponding to the subject's state. In some embodiments, the machine learning model 218 is trained using the secondary dataset to generate a walking gait analysis signal corresponding to a subject's state.

The touch signal is relayed to a lower limb prosthesis. The lower limb is configured to elicit a subject movement response. In an example, the subject movement response includes a movement of a foot of the lower limb prosthesis. In some examples, the touch signal is also communicated from the lower limb prosthesis to the haptic feedback system 222. In an example, the haptic feedback system 222 includes a vest worn on the subject's chest. In other examples, the haptic feedback system 222 may be a hand worn band, or such wearable devices. The haptic feedback system 222 elicits a haptic response corresponding to the subject's state. In an examples, the haptic response may include vibration, motions, and/or forces that provide a sense of touch to the subject.

### Examples and Experiments

Experiments were conducted using various classification and machine learning (ML) models to classify and predict EEG signals from a set of experiments involving participants wearing the BCI 202 and the FBG sensor 206_{1-N} installed in the wearable sandal arrangement 226. The experiments were performed in two parts namely Part I-BCI Classification and Part II-BCI Prediction. The purpose of the first part of the experiment is to identify the electrodes on the BCI 202 that are more responsive to foot movement. Later in the second part, EEG signals are predicted against a random plantar pressure information. The machine learning models was implemented using Python and pre-processing of the data was performed using MATLAB. For clarity, the experiments and implementation is presented in two parts namely: BCI Classification and BCI Classification Experiment.

### BCI Classification:

In this part, the collection of EEG data was performed from the BCI 202. The EEG data classification was performed on the incoming data from the BCI device in the a. sitting, b. standing, and c. walking states of the participants. The EEG data was processed to identify the EEG signals directly associated with the foot movement with a reduced number of channels compared to the original 16 channels.

### BCI Classification Experiment:

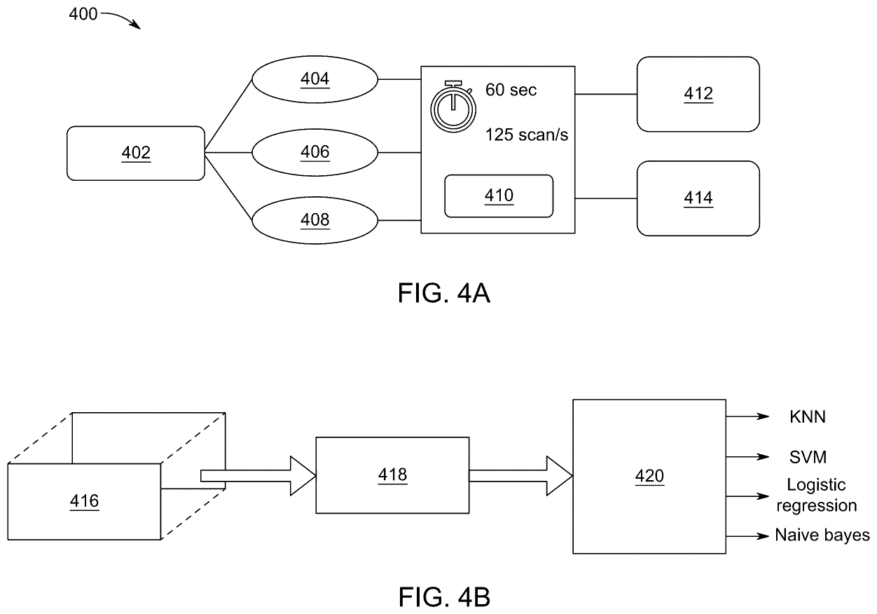

The experimental setup consists of wearing the BCI device and performing the three gait positions, where two participants were made to sit in a chair of height 50 cm and with feet resting on the ground. For standing, while maintaining a good posture, the participants were made to stand with minimal movement. Finally, the walking gait was recorded by making the participants walk in a straight path for 60 seconds. The schematic for the experiment is shown in FIG. 4A. FIG. 4A illustrates a BCI classification stage, according to certain embodiments

During the first step, the participants were equipped with the BCI 202 and performed a plurality of gait positions or state such as sitting position 404, standing position 406 and walking position 408. For example, the participants were initially in sitting position with the BCI 202 head-mounted. A block 402 indicates an illustrative representation of 16 BCI channels or electrodes of the BCI 202 on the head of the participants. When the participants were in sitting state, a leg was resting on the ground. In the sitting state of the participants with a foot on the ground, plantar pressure from the foot was applied to the ground. Based upon the plantar-plantar pressure of the subject, corresponding brain signals were generated in the brain of the subject. A plurality of the EEG signals corresponding to brain signals of the participants were detected in 16 channels of the BCI 202. Each signal of the plurality of EEG signals was registered by corresponding channels of the plurality of 16 channels of the BCI 202. The generated EEG signals were received by the controller 256 of the BCI 202 in 1^{st} trial through 16 different channels. In an aspect, the participants were made to perform the state for at least 10 trails. The block 410 illustrates the number of trials performed by the subject where each trial was performed for at least 60 seconds. In an example, the controller 256 was configured to scan the generated EEG signals at 125 scan/sec. In some examples, the number of trails and the scanning speed of the generated EEG signals were higher or lower than 125 scans/sec. Similarly, the controller 256 collected the EEG signals for the participant in a standing state and a walking state.

In the experiment, the computer controller 210 was used to create a machine learning model using the data obtained from the three states of the subject. The data was split into a training and testing set in 80-20 ratio. A block 412 shows a participant's data for the training set whereas a block 414 shows the participant's data for testing set when the model is trained using 80% of the data.

FIG. 4B illustrates a flow diagram of data classification in the computer controller 210. The computer controller 210 used a plurality of classification models such as K-Nearest Neighbor (KNN), Support Vector Machine (SVM), Logistic Regression (LR), and Naïve Bayes (NB). In a normal analysis of the EEG data, the difference between sitting position, standing position and walking position may not be apparent in plurality of channels, since the brain signals may not have a one-to-one mapping to sensory perception of subjects or participants. Therefore, it may be difficult to use machine learning models as the signal was composed of different factors. However, using plurality of classification models, the accuracy was improved. For example, the computer controller 210 used KNN, and NB to obtain the high accuracy in detecting the plantar pressure response. In some examples, the computer controller 210 used other classification models as well.

Block 416 refers to the collected data sampled over 16 BCI channels from the BCI 202. Collected dataset was preprocessed using a preprocessing block 418 of the computer controller 210. Since, the classification dataset was recorded from at least two participants for three different gait states: the sitting state, the standing state, and the walking state, the brain activity was collected for each participant for ten trials, each for 60 seconds in the three gait positions. Each trial has data from 16 electrodes sensing the brain activity, which provides 16 signals in each trial. Hence, the total number of trials was found to be 59. Also, each signal had 7,500 data points since the sampling frequency was 125 Hz. Furthermore, the data were reorganized such that all the signals from all trials for one electrode were in one data file, resulting in 16 data files. Hence, the brain activity signal was used as the only feature for the classification model. The output variable of the data set is the gait's position; therefore, these outputs were encoded as 0 for sitting, 1 for standing, and 2 for walking.

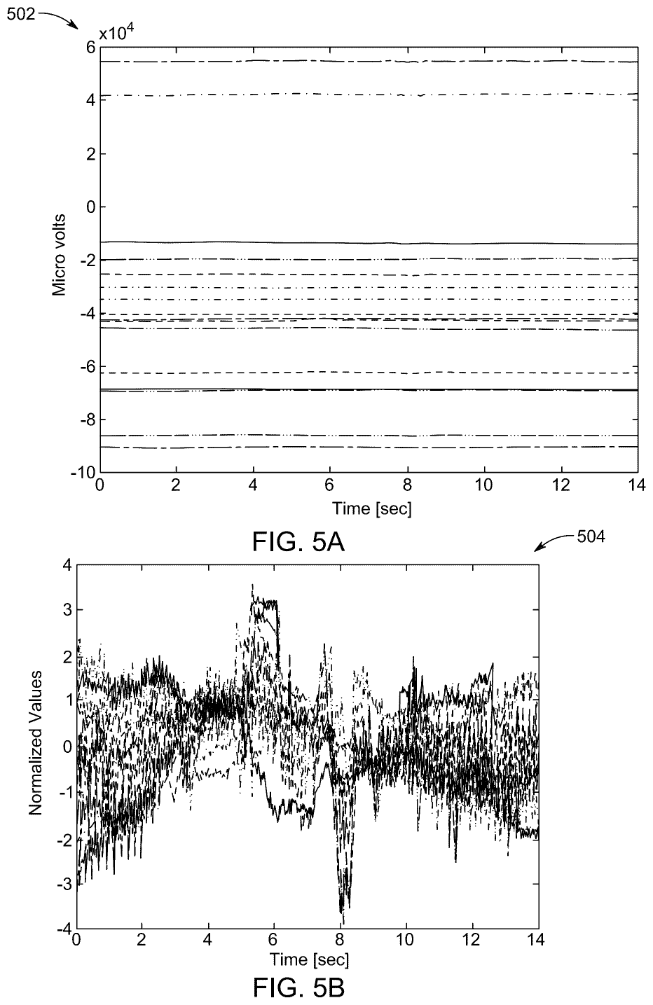

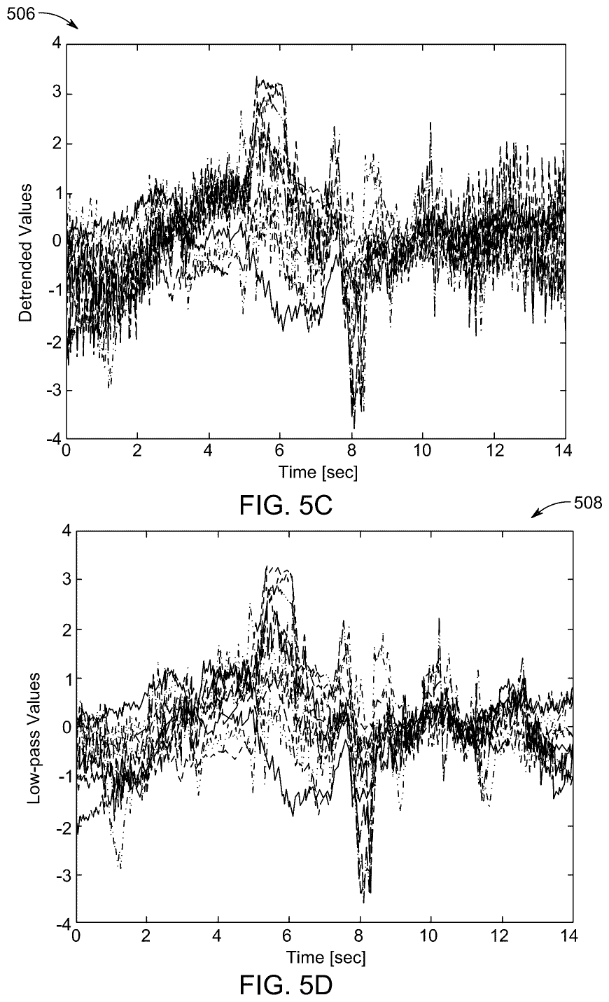

FIG. 5A illustrates raw EEG data 502 of the 16 channels during walking of a single participant, according to an embodiment. When the participant walks, sits or stands, each channel detects generation of electric signals in the electrodes 272 of the BCI 202. The preprocessing block 418, after receiving the EEG signal dataset over 16 channels, preprocessed the dataset by removing the first and last 5 seconds of the data for stability purposes.

FIG. 5B illustrates a graph 504 as a normalized value of the preprocessed EEG dataset, according to an embodiment. The preprocessing block 418, after removing the first and last 5 seconds for stability purpose, detrended (involving removing a mean of each channel), normalized using a z-score method, and low passed at 60 Hz to remove 60 Hz mains noise. From the EEG signal data, the difference between sitting and walking was not apparent in some channels, indicating that brain signals did not have a one-to-one mapping to sensory perception. As a result, it was harder to classify or use machine learning models since the signal is a composition of different factors. However, several models were successful in obtaining accuracy including K-NN and Naïve Bayes, for the channels that are relatively sensitive to plantar pressure. To denoise the EEG signal, the preprocessing block 418 used two methods. In a first method, the resulting signal was normalized followed with detrending and application of a low pass filter. In a second method, detrending was applied using wavelet decomposition. These methods were a part of the preprocessing operations. The normalizing operation involved normalizing the EEG signal using the z-score so the magnitude difference between the tests would be negligible. The z-score provided an indication of how far a datapoint is from the mean a data point. The preprocessing block 418 performed the normalization process so that the magnitude difference between the tests became negligible. The normalization process also resulted in removing the DC offset of the channels. Instead of using a high-pass filter at 0.1 Hz, detrending was used which removed the mean of each channel. Detrending was used since it could improve the accuracy of the machine learning. The low-pass filter was applied post detrending to remove the line noise at 60 Hz.

FIG. 5C illustrates a graph 506 as a detrended value of the preprocessed EEG signal dataset, according to an embodiment. Once, normalization process is done, the preprocessing block 418 detrends the normalized data. The detrending process removes the mean of each channel as well as helps improving the accuracy of the classification model.

FIG. 5D illustrates a graph 508 of the processed EEG signal dataset after passing the detrended value with a low pass filter, according to an embodiment. Once the detrending process is done, the preprocessing block 418 uses a low pass filter to remove the line noise at 60 Hz.

Although the graph in FIGS. 5A, 5B, 5C and 5D is illustrated, as an example, for walking state of the subject or participant, similar graph for other state of the subject, that is, sitting and standing is also retrieved, normalized, detrended and passed through low pass filtering process to generate a preprocessed dataset for plurality of classification models.

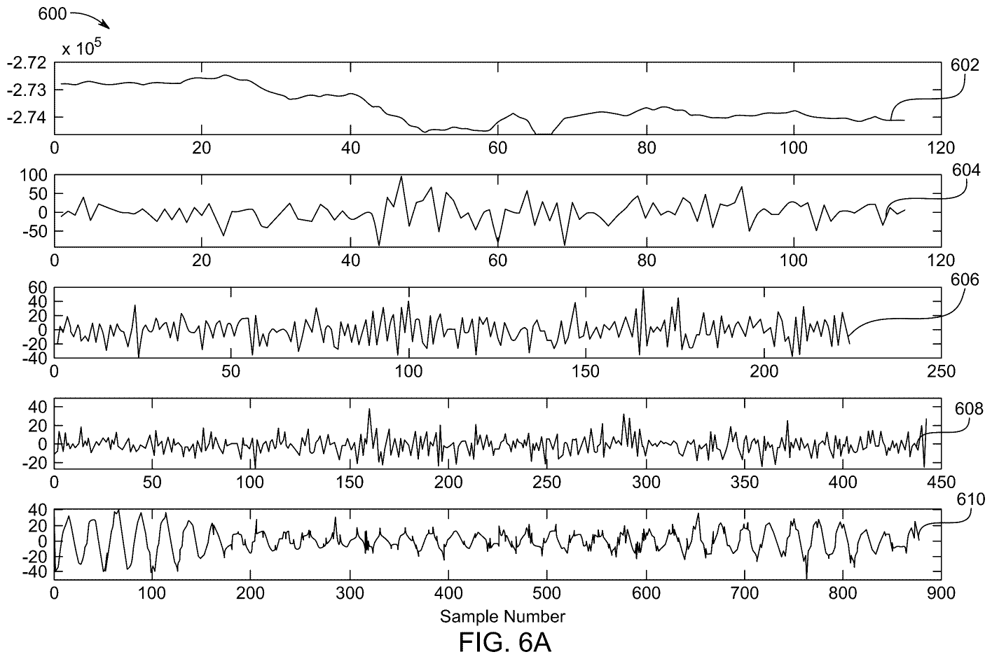

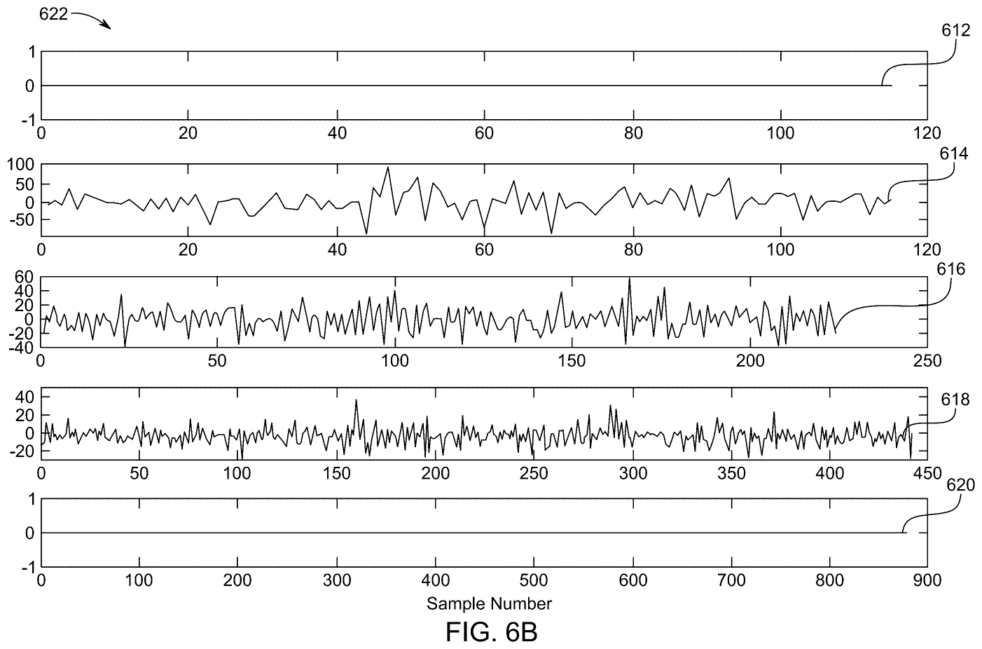

FIG. 6A illustrates a 4-level wavelet decomposition pattern 600 of 16 channels, according to an embodiment. The preprocessing block 418 further uses a second method for detrending EEG signal dataset in a more effective way. The preprocessing block 418 applied a wavelet transform of the EEG signal dataset over 16 channels after removing the first and last 5 seconds of the data. A graph 602 illustrates a pattern of approximation of coefficients. A graph 604 illustrates a pattern of level 4 detail coefficients. A graph 606 illustrates a pattern of level 3 coefficients. A graph 608 illustrates a pattern of level 2 detail coefficients and a graph 610 illustrates a pattern of level 1 detail coefficients. The level 1 detailed coefficient contains the 60 Hz line noise, while the approximation contains the low-frequency trend of the signal.

FIG. 6B illustrates a 4-level wavelet decomposition pattern 622 of 16 channels with removed level 1 and the approximation of coefficients, according to an embodiment. The block 418 removes the trend and the line noise by removing level 1 coefficients and the approximation of coefficients, such that the values of signals at these level are zero. Accordingly, a graph 612 and a graph 620 attains zero value whereas a graph 614, 616 and 618 are similar to the graph 604, 606 and 608, respectively.

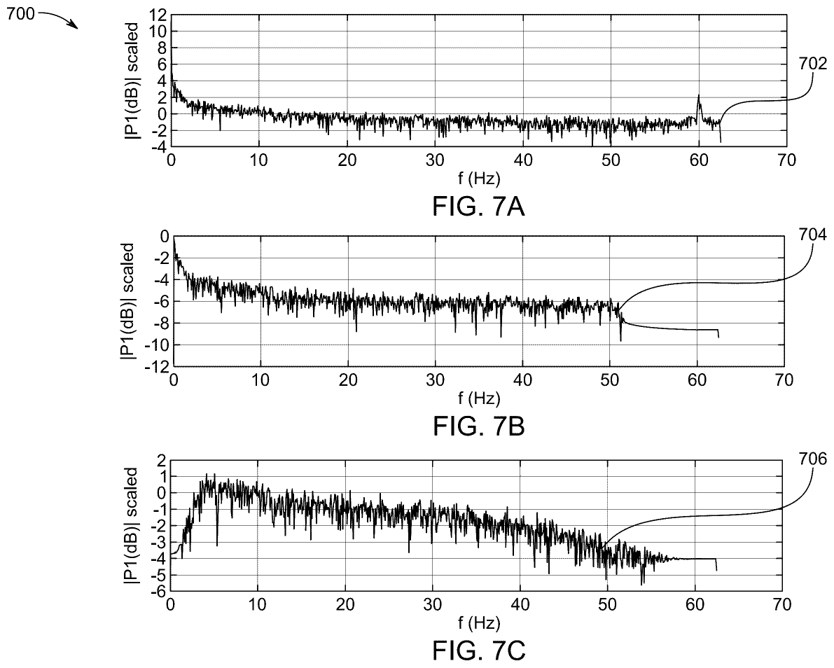

FIG. 7 illustrates a Fourier transform pattern 700 of the raw signal, processed signals using the first method and the processed signal using the second method. A graph 702 illustrates a single sided amplitude spectrum of the raw signal received at 16 channels of the BCI 202. A graph 704 illustrates a single sided amplitude spectrum of the preprocessed signal from the raw signals received at 16 channels of the BCI 202. A graph 706 illustrates a single sided amplitude spectrum of the wavelet transform signal from the raw signals received at 16 channels of the BCI 202.

Referring back to FIG. 4, the preprocessed EEG signals including the EEG signals were preprocessed using the first method as well as the second method and the raw data is provided to a block 420 of the computer controller 210. The block 420 is configured for providing the preprocessed EEG signals and the raw data signals to plurality of classification models for training. The classification models are KNN, SVM, Logistic regression (LR) and Naïve bayes (NB). As disclosed earlier, 80% of the preprocessed EEG signal and the raw data is used for training and 20% is used for testing each model for identifying the accuracy of the model. Also, each electrode datafile has its own classifier using different techniques.

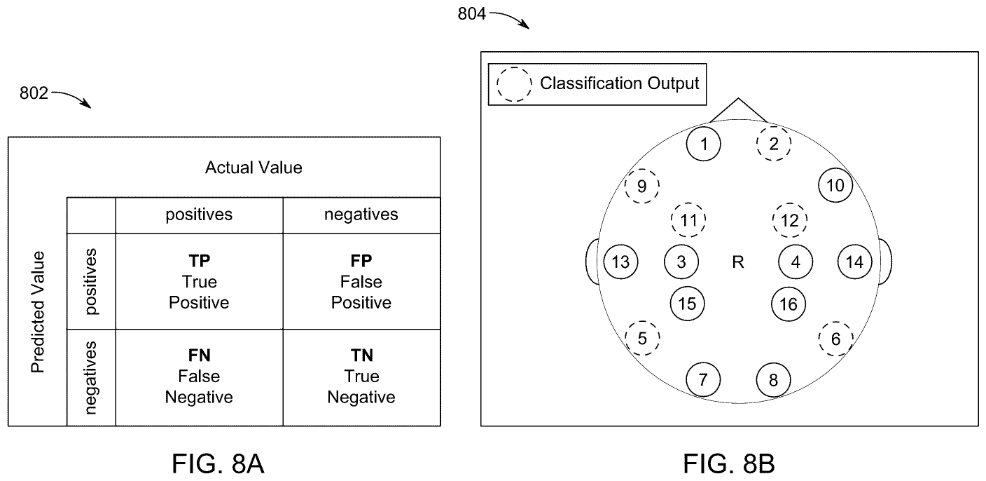

FIG. 8A illustrates a confusion matrix 802 based on results of predicted value and the actual value of each model, according to an embodiment. Based upon the elements of the confusion matrix, the computer controller 210 was configured to measure performance of each model. The performance measure for the experiment was accuracy and balanced accuracy. The accuracy is the ratio between the number of the current prediction to the total number of predictions and is given by:

$$Accuracy = \frac{TP + TN}{TP + FP + TN + FN} , \qquad ( 1 )$$

where, TP=true positive,
TN=True negative,
FP=False positive, and
FN=False negative.

Similarly the balanced accuracy is given by:

$$Balanced Accuracy = \frac{Sensitivity + Specificity}{2} , \qquad ( 2 )$$

$$where ,$$

$$Sensitivity = \frac{TP}{TP + FN} , \qquad ( 3 )$$

and specificity is given by:

$$Specificity = \frac{TP}{TP + FN} ; \qquad ( 4 )$$

Each of the classification model or the classifier uses the raw data and the processed sampled data and provides the accuracy result for each of the plurality of 16 channels, as given in Table 1:

TABLE 1
Accuracy results of channel classification.

| | SAMPLED | | | | | | | | | | | | | | | |
|---|---|---|---|---|---|---|---|---|---|---|---|---|---|---|---|---|
| | K-NN | | | | SVM | | | | Logistic regression | | | | [symbol] | | | |
| CH | TR | VAL | T | [symbol] | TR | VAL | T | [symbol] | TR | VAL | T | [symbol] | TR | VAL | T | [symbol] |
| 1 | 1 | 0.81 | 0.75 | 0.8 | 0. [symbol] | 0. [symbol] | 0.75 | 0. [symbol] | 1 | | 0. [symbol] | 0. [symbol] | 0.72 | 0. [symbol] | 0. [symbol] | 0.5 |
| 2 | 0.74 | 0.74 | 0.92 | [symbol] | 0. [symbol] | 0. [symbol] | 0. [symbol] | 0. [symbol] | 1 | 0.55 | 0.25 | 0.24 | 0.74 | 0.72 | 0. [symbol] | 0. [symbol] |
| 3 | 1 | 0. [symbol] 8 | 0.5 | [symbol] | 0.78 | 0. [symbol] | 0.42 | 0.4 | 1 | 0. [symbol] | 0.42 | 0.44 | 0.7 | 0. [symbol] | 0. [symbol] | 0.62 |
| 4 | 1 | 0.59 | 0.75 | 0.78 | 0.98 | 0.73 | 0.75 | 0. [symbol] | 1 | 0. [symbol] | 0. [symbol] | 0. [symbol] | 0.55 | 0.53 | 0.5 | 0. [symbol] |
| 5 | 0.81 | 0.77 | 0.83 | [symbol] | 1 | 0. [symbol] | 0.5 | 0. [symbol] | 1 | 0.49 | 0. [symbol] | 0. [symbol] | 0. [symbol] | 0. [symbol] | 0. [symbol] | 0.67 |
| 6 | 1 | 0.53 | 0.83 | [symbol] | 1 | 0.42 | 0.42 | 0.5 | 1 | 0.4 [symbol] | 0. [symbol] | 0. [symbol] | 0.51 | 0.51 | 0. [symbol] | 0.6 |
| 7 | 1 | [symbol] | 0.58 | 0.59 | 1 | [symbol] | [symbol] | 0. [symbol] 7 | 1 | 0.4 [symbol] | 0. [symbol] | 0. [symbol] | 0. [symbol] | 0.6 | 0. [symbol] | 0.64 |
| 8 | 1 | [symbol] | 0.67 | 0.67 | 1 | 0.42 | 0.42 | 0.47 | 1 | 0. [symbol] | 0.25 | 0. [symbol] | 0. [symbol] | 0.49 | 0. [symbol] | 0. [symbol] |
| 9 | 1 | 0.6 | 0.9 [symbol] | [symbol] | 1 | [symbol] | 0.25 | 0. [symbol] | 1 | 0. [symbol] | 0. [symbol] | 0. [symbol] | 0.55 | 0. [symbol] | 0.58 | 0. [symbol] |
| 10 | 1 | 0.54 | 0.58 | 0.59 | 1 | 0.25 | [symbol] | 0.42 | 1 | 0.62 | 0. [symbol] | 0. [symbol] | 0.55 | 0. [symbol] | 0.58 | 0. [symbol] |
| 11 | 1 | 0.54 | 0.5 | 0.4 [symbol] | 1 | [symbol] | 0.5 | 0.57 | 1 | 0.32 | 0. [symbol] | 0. [symbol] | 0. [symbol] | 0. [symbol] | 0.67 | 0. [symbol] |
| 12 | 0.74 | 0.7 | 0.5 | 0.51 | 1 | 0.47 | 0.5 | 0. [symbol] 8 | 1 | 0. [symbol] | 0. [symbol] | 0. [symbol] | 0.57 | 0. [symbol] | 0.42 | 0. [symbol] |
| 13 | 1 | 0.47 | 0.42 | 0.47 | 1 | [symbol] | 0.25 | [symbol] | 1 | 0. [symbol] | 0. [symbol] | 0. [symbol] | 0.51 | 0. [symbol] | 0. [symbol] | 0. [symbol] |
| 14 | 1 | 0.55 | 0.6 [symbol] | 0.67 | 1 | [symbol] | [symbol] | 0.4 | 1 | 0. [symbol] | 0.08 | 0. [symbol] | 0.51 | 0. [symbol] | 0. [symbol] | 0. [symbol] |
| 15 | 1 | 0.69 | 0.67 | 0.6 [symbol] | 1 | 0.42 | [symbol] | 0.42 | 1 | 0.42 | 0. [symbol] | 0. [symbol] | 0.57 | 0.55 | 0.42 | 0.4 |
| 16 | 1 | 0. [symbol] | 0.5 | 0.51 | 1 | 0.44 | [symbol] | 0.47 | 1 | 0.4 [symbol] | 0. [symbol] | 0. [symbol] | 0.32 | 0.73 | 0.42 | 0. [symbol] |
| Avg | 0.96 | 0. [symbol] | [symbol] | [symbol] | [symbol] 7 | 0.47 | [symbol] | 0.49 | 1 | 0. [symbol] | 0. [symbol] | 0. [symbol] | 0.57 | 0. [symbol] | 0. [symbol] | 0. [symbol] |

| | PROCESSED | | | | | | | | | | | | | | | |
|---|---|---|---|---|---|---|---|---|---|---|---|---|---|---|---|---|
| | K-NN | | | | SVM | | | | Logistic regression | | | | [symbol] | | | |
| CH | TR | VAL | T | [symbol] | TR | VAL | T | [symbol] | TR | VAL | T | [symbol] | TR | VAL | T | [symbol] |
| 1 | 1 | 0. [symbol] | 0. [symbol] | 0. [symbol] | 1 | 0. [symbol] | 0.5 | 0. [symbol] | 1 | 0. [symbol] | 0.25 | 0. [symbol] | 0.81 | 0.56 | 0. [symbol] | 0. [symbol] |
| 2 | 1 | 0.45 | 0.17 | 0.22 | 1 | 0.43 | 0.25 | 0.29 | 1 | 0. [symbol] | 0.25 | 0. [symbol] | 0.68 | 0.55 | 0.58 | 0.6 |
| 3 | 1 | 0.51 | 0.5 | 0.58 | 1 | 0.44 | 0.42 | 0.5 | 1 | 0. [symbol] | 0.25 | 0. [symbol] | 0. [symbol] | 0.51 | 0. [symbol] | 0. [symbol] |
| 4 | 1 | 0.49 | 0.57 | 0.67 | 1 | 0.32 | 0. [symbol] | 0.72 | 1 | 0.25 | 0.75 | 0. [symbol] | 0.68 | 0.62 | 0.75 | 0.67 |
| 5 | 1 | 0. [symbol] | 0.58 | 0.53 | 1 | 0. [symbol] | 0. [symbol] | 0. [symbol] | 1 | 0. [symbol] | 0.75 | 0.76 | 0.62 | 0. [symbol] | 0.75 | 0.75 |
| 6 | 1 | 0. [symbol] | 0. [symbol] | 0.42 | 1 | 0.47 | 0.42 | 0. [symbol] | 1 | 0.4 | 0.42 | 0.47 | 0.74 | 0. [symbol] | 0.83 | 0. [symbol] |
| 7 | 1 | 0.45 | 0. [symbol] | 0.42 | 1 | 0.44 | 0.42 | 0.29 | 1 | 0. [symbol] | 0. [symbol] | 0. [symbol] | 0. [symbol] | 0.5 [symbol] | 0. [symbol] | 0. [symbol] |
| 8 | 1 | 0. [symbol] | 0. [symbol] | 0. [symbol] | 1 | 0.34 | 0. [symbol] | 0.47 | 1 | 0.32 | 0.42 | 0.5 | 0. [symbol] | 0.64 | 0.5 | 0.48 |
| 9 | 1 | 0.49 | 0. [symbol] | 0.24 | 1 | 0.32 | 0. [symbol] | 0.28 | 1 | 0. [symbol] | 0. [symbol] | 0. [symbol] | 0.74 | 0.53 | 0. [symbol] | 0. [symbol] |
| 10 | 1 | 0.42 | 0.25 | 0. [symbol] | 1 | 0. [symbol] | 0. [symbol] | 0. [symbol] | 1 | 0.27 | 0. [symbol] | 0.4 | 0. [symbol] | 0.59 | 0.42 | 0. [symbol] |
| 11 | 1 | 0.47 | 0.42 | 0.5 | 1 | 0.45 | 0. [symbol] | 0. [symbol] | 1 | 0.29 | 0.42 | 0.46 | 0.7 | 0.53 | 0. [symbol] | 0. [symbol] |
| 12 | 1 | 0. [symbol] | 0.5 | 0. [symbol] | 1 | 0.51 | 0.58 | 0.58 | 1 | 0. [symbol] | 0.5 | 0.58 | 0. [symbol] | 0.7 | 0.83 | 0. [symbol] |
| 13 | 1 | 0.45 | 0.5 | 0.57 | 1 | 0. [symbol] | 0.57 | 0.47 | 1 | 0. [symbol] | 0. [symbol] | 0. [symbol] | 0. [symbol] | 0. [symbol] | 0.42 | 0. [symbol] |
| 14 | 1 | 0. [symbol] | 0. [symbol] | 0.48 | 1 | 0. [symbol] | 0. [symbol] | 0. [symbol] | 0. [symbol] | 0.28 | 0.58 | 0.67 | 0.83 | 0.45 | 0. [symbol] | 0. [symbol] |
| 15 | 1 | 0. [symbol] | 0.58 | 0. [symbol] | 1 | 0. [symbol] | 0.66 | 0.58 | 1 | 0.28 | 0.42 | 0. [symbol] | 0.8 | 0.49 | 0.5 | 0.51 |
| 16 | 1 | 0. [symbol] | 0.42 | 0.47 | 1 | 0.42 | 0.47 | 0.47 | 1 | 0. [symbol] | 0.25 | 0. [symbol] | 0.77 | 0. [symbol] | 0.5 | 0. [symbol] |
| Avg | 1 | 0.46 | 0.41 | 0.46 | 1 | 0.41 | 0.46 | 0. [symbol] | 1 | 0. [symbol] | 0.4 | 0.45 | 0.74 | 0.54 | 0. [symbol] | 0. [symbol] |

[symbol]

indicates data missing or illegible when filed

Based upon the accuracy, as provided in Table 1, it was found that K-NN classifier performs better on the raw data, whereas NB classifier performs better on the processed data. Also, gait posture influences channels 2, 5, 6, and 9 were found to have a better accuracy than the rest of the channels, channel 2, 5, 6 and 9 are found to be responding more for any state of the subject even if data is raw. Similarly, the data affected by the gait posture has smaller variance. Accordingly, channels 6, 9, 11 and 12 are found to be responding more for any state of the subject if processed data is used. As such, the computer controller 1010 selects a subset of the channels that includes 6 channels that is, channel no. 2, 5, 6, 9, 11 and 12, in order to include all possibility of signals. In other words, channels 2, 5, 6, 9, 11 and 12 are sensitive to any gait posture from different perspectives to ensure all possibilities. For example, if a subject is in the sitting state, standing state or in walking state, channels 2, 5, 6, 9, 11 and 12 are identified as the channels that receives maximum EEG signals.

FIG. 8B illustrates a graphical representation of channels identified as most sensitive to state of the subject 804, according to an embodiment. Based upon the subsections of the channels that is, channels 2, 5, 6, 9, 11 and 12, the second part of the experiment for predicting the brain signals is performed that considers only the aforementioned channels. Considering only the selected subset of channels ease the burden of computation on the computer controller 210 while predicting the brain signal and thus the touch signals.

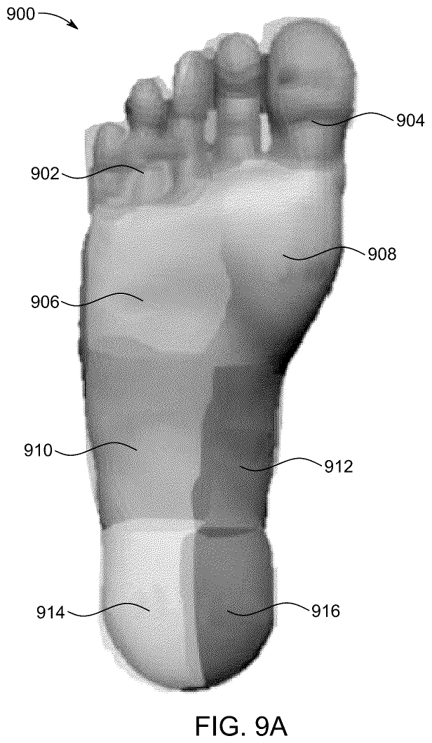

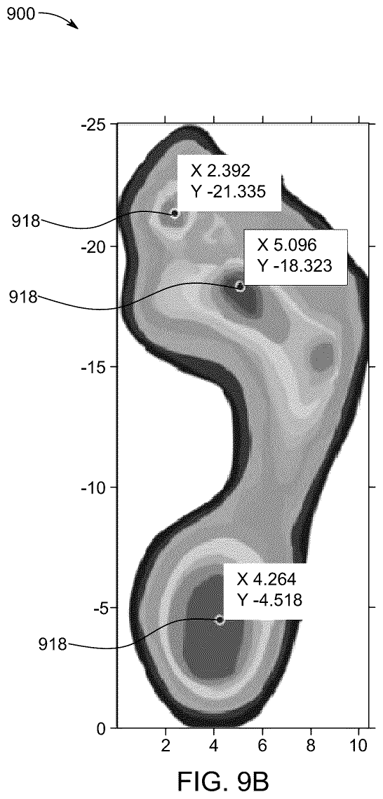

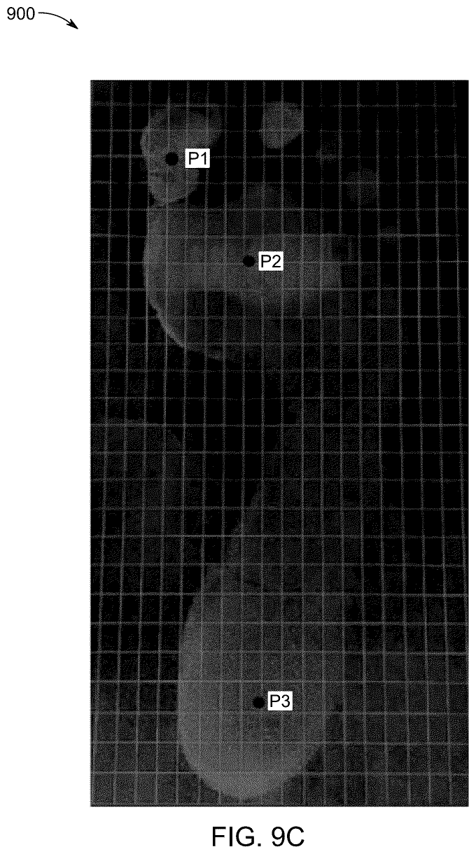

When the first part of the experiment i.e., identifying the number of electrodes most responsive to the subject state, was done, the second part of the experiment was initiated that included predicting touch signals based upon the plantar pressure information of the subject or participant. In order to perform the second part of the experiment, foot geometry and plantar pressure measurement using a plurality of sensors arranged in sole was performed. Accordingly, FIG. 9A illustrates geometry of a foot sole 900 of a normal leg, according to an embodiment. Mapping an accurate ground contact is important to identify an accurate ground contact scenario in plurality of states of the subject. The plurality of states are sitting state, standing state and walking state. The ground contact of the foot sole 900 and pressure distribution may be identified using plantar pressure measurement in key regions of the foot sole 900 using plurality of sensors in plurality of states of the subject. The foot sole 900 may be segmented into plurality of regions such as a first region 902 as lesser toes, a second region 904 as hallux, a third region 906 as lateral forefoot, a fourth region 908 as medial forefoot, a fifth region 910 as lateral mid foot, a sixth region 912 as medial midfoot, a seventh region 914 as lateral rearfoot and an eighth region 916 as medial rearfoot.

FIG. 9B illustrates points of the foot sole 900 identified as critically imperative points for plantar pressure measurement, according to an embodiment. A MATLAB representation of the foot sole 900 shows at least three (3) critical points 918 identified as to bear maximum plantar pressure in all three states of the subject.

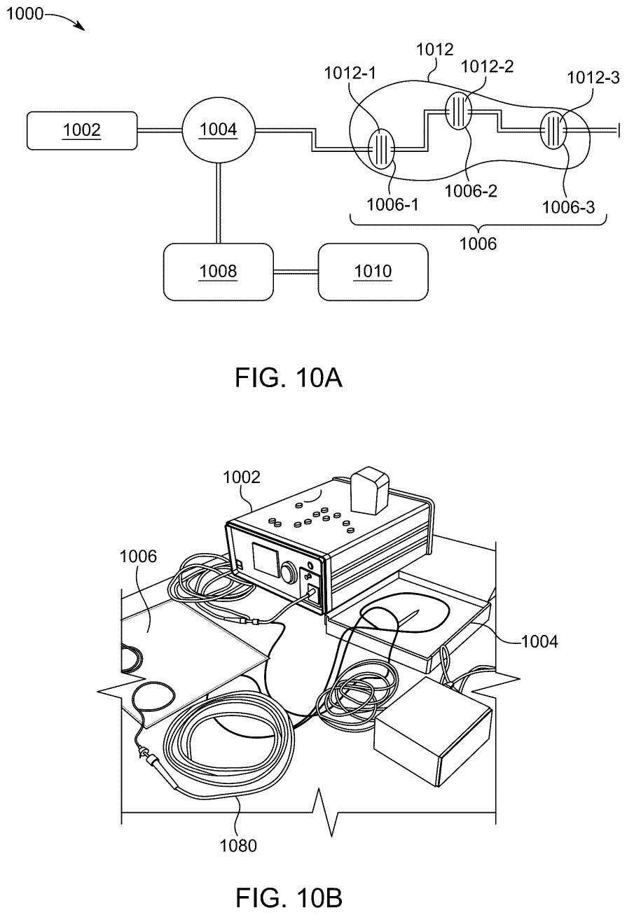

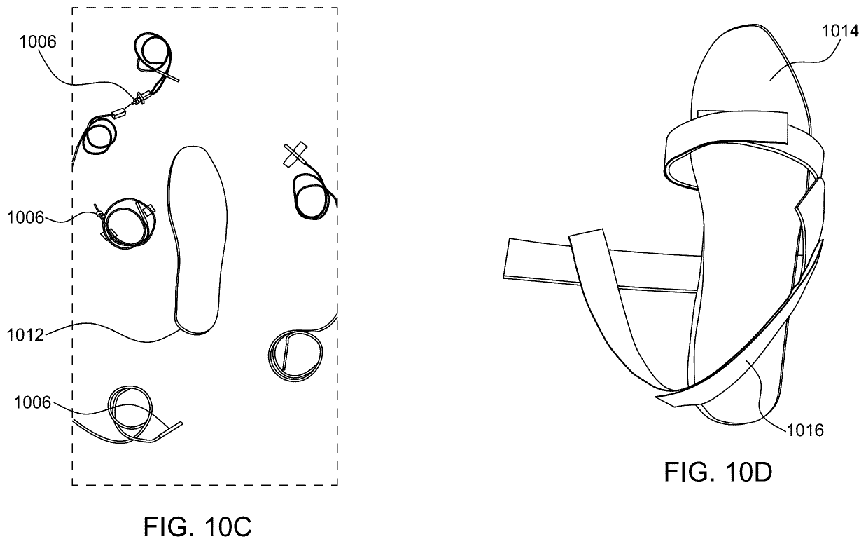

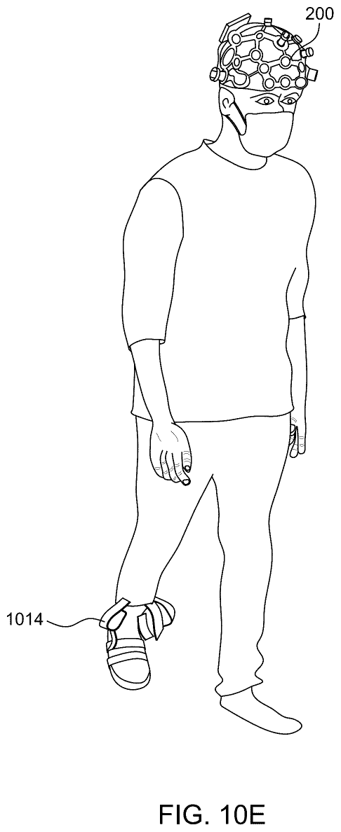

FIG. 9C further illustrates foot pressure identified with markings on an ink pad, according to an embodiment. A pressure map of the foot sole 900 is created with the ink pad shows critical points on the foot sole 900 for measuring plantar pressure. Accordingly, identified pressure points are used as sensors positioning in a sole of a shoe as illustrated in FIG. 10.

FIG. 10A illustrates a walking gate analysis apparatus 1000, according to an embodiment. The walking gate analysis apparatus 1000 includes plurality of sensors 1006, the BCI 202 and the computer controller 1010 (same as the computer controller 210). Plurality of sensors 1006 are disposed on a toe 1012-1, a midfoot 1012-2, and a heel 1012-3 of an insole 1012. The location of placement of plurality of sensors 1006 are identified as the plantar pressure points or critical points 918 in FIG. 9. The plurality of sensors 1006 are FBG sensors of size 250 μm. The FBG sensors 1006 are based on optical fiber technology, where a specific region in the fiber ranging from 10 mm to 20 mm along fiber length, is etched by ultraviolet (UV) radiations to produce gratings with a specific period. Plurality of sensors 1006 include a first FBG sensor 1006-1, a second FBG sensor 1006-2 and a third FBG sensor 1006-3. The first FBG sensor 1006-1 is fixed to the toe 1012-1, the second FBG sensor 1006-2 is fixed on the midfoot 1012-2 and the third FBG sensor 1006-3 is fixed on the heel 1012-3 of the insole 1012. In an embodiment, each of the plurality of sensors 1006 is fixed to at least one of the eight distinct regions as described in FIG. 9A. Also, each of the plurality of sensors 1006 is not fixed to the same distinct region out of 8 identified regions. As such no two sensors are placed in the same region of the foot sole 900.

The walking gate analysis apparatus 1000 includes a broadband light source 1002 of wavelength 1510-1590 nm. In an embodiment, the broadband light source could be a portable light source 1002. In another embodiment, the light source could be a laser light 1002. In another embodiment, the light source 1002 may have multiple wavelengths. The walking gate analysis apparatus 1000 includes an optical circulator 1004. The optical circulator 1004 includes at least three input ports. The optical circulator 1004 is configured to receive input light at one of the port from the broadband light source 1002 and transmit the light signal on immediate next port that is, to the FBG sensors 1006. The basic working principle of the optical circulator 1004 is explained hereafter. A first port, for example, receives a light signal from the light source 1002. The optical circulator 1004 is optically connected to another devices, such as FBG sensors 1006 at a second port, for example. When the light signal is transmitted from the light source towards the first port of the optical circulator 1004, the optical circulator 1004 is transmitted to the second port of the optical circulator 1004 towards the FBG sensors 1006. However, if FBG sensors reflect some of the light back, the reflected light is received in the third port of the optical circulator 1004. As such, the circulation of the light signal in the optical circulator 1004 may be in a clockwise direction or anticlockwise direction, depending upon a configuration of the optical circulator 1004. Accordingly, the optical circulator 1004 provides a three-way gateway between the light source 1002, an optical interrogator 1008, and the plurality of FBG sensors 1006. The optical interrogator 1008 is discussed in detail in the next section.

The walking gate analysis apparatus 1000 includes the optical interrogator 1008. The reflected light signal may be observed on the optical interrogation monitor. The optical interrogator 1008 is optically connected to the third port of the optical circulator 1004. The interrogation 1008 may include a display unit. In an embodiment, the optical interrogation monitor is IMON USB 512 produced by IBSEN Photonics (See: Ibsen Photonics A/S, Ryttermarken 17, DK-3520 Farum, Denmark). Further, the information about the reflected signal, also known as the wavelength signal, is transmitted to the computer controller 1010 for identifying the distribution of plantar pressure of the subject.

FIG. 10B illustrates a physical setup of the walking gate analysis apparatus 1000 of FIG. 10A, including the light source 1002, the FBG 1006, the optical interrogator 1080m and the optical circulator 1004, according to an embodiment.

FIG. 10C illustrates the insole 1012 and the sensor setup in the insole 1012, according to an embodiment. The sensors 1006 may, initially be coated with a protective layer to protect from harsh environments, such as mechanical impact. In an embodiment, the sensor 1006 may be coated with heat-shrinkable fusion splice protection sleeve. In another embodiment, the sensor 1006 may be coated with metal such as nickel using electroless-electro plating method. In another embodiment, the sensor 1006 may be coated with carbon-coated fibers. Any material for coating layer may be selected that is currently known in the art. Once the coating process is complete, the sensors 1006 may be glued on the insole 1012 in a fixed position.

FIG. 10D illustrates a wearable sandal arrangement 1014, according to an embodiment. Once the sensors 1006 are glued at an appropriate position in the insole 1012, the insole 1012 is fixed underside of the wearable sandal arrangement 1014, in such a way that under pressure of the foot of a wearer, the sensors does not misplace from their initial location. A Velcro strap-on 1016 is fixed using, a glue or a permanent stitch, for example, on the top of the wearable sandal arrangement 1014. The Velcro strap-on 1016 tightens the wearable sandal arrangement 1014 with the leg of the wearer at the time of performing the experiment.

FIG. 10E shows an exemplary illustration of a subject wearing the wearable sandal arrangement 1014 along with the BCI 202 on his head for predicting the brain signals and/or touch signal, according to an embodiment. During the second part of the experiment, the subject wears the BCI 202 as well as the wearable sandal arrangement 1014 on his foot.

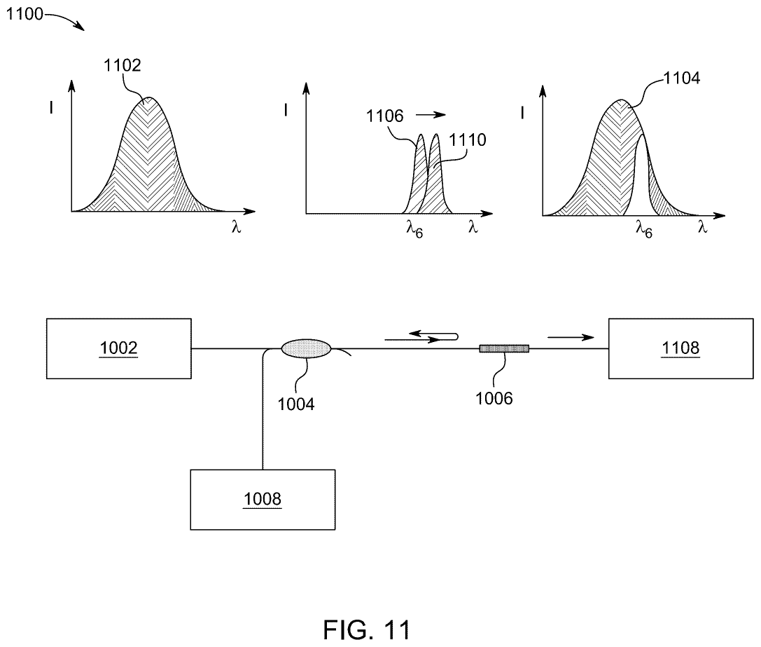

FIG. 11 illustrates a diagram 1100 showing the working principle of the FBG sensor 1006, according to an embodiment. Each of the FBG sensor 1006 possesses a base wavelength or a unique wavelength signal. The base wavelength or the unique wavelength signals determines the wavelength of the signal that would be reflected when a light is input to the FBG sensor 1006. The concept of the wavelength reflection in FBG sensor 1006 is now discussed in more detail. The FBG sensors 1006 are manufactured in such way that whenever a light source 1002 projects a light signal having a first spectrum 1102 to the FBG sensor 1006 through the optical circulator 1004, the light signal with a second spectrum 1104 is transmitted through the FBG sensor 1006 received though a collector 1108. However, a signal of a specific wavelength, also known as a Bragg wavelength or the base wavelength or a baseline wavelength as a third spectrum 1106, is reflected back from the FBG sensor 1006 towards the light source 1002 via the optical circulator 1004. Since the optical circulator 1004 transmits any incoming light on the next port, the interrogation monitor or the optical interrogator 1008 optically connected on the next port of the optical circulator 1004 receives the reflected light signal where the base wavelength reflected signal may be observed and analysis may be made based upon the reflected signals. The base wavelength or the baseline wavelength for the first FBG sensor 1006-1, the second FBG sensor 1006-2 and the third FBG sensor 1006-3 are 1545.341 nm, 1535.068 nm and 1539.966 nm, respectively. Accordingly, wavelength corresponding to the 1545.341 nm, 1535.068 nm and 1539.966 nm are reflected back to the optical circulator 1004.

In an embodiment, a pressure is applied over FBG sensors 1006-1, 1006-2 and 1006-3, a shift in wavelength in the reflected signal is observed. As such, a fourth spectrum 1110 is observed in the interrogation monitor or the optical interrogator 1008 on application of pressure over the FBG sensors 1006. The shift in wavelength is directly proportional to the applied pressure on the FBG, that is, more is the pressure over the FBG sensor 1006, more is the shift in wavelength in the reflected signal.

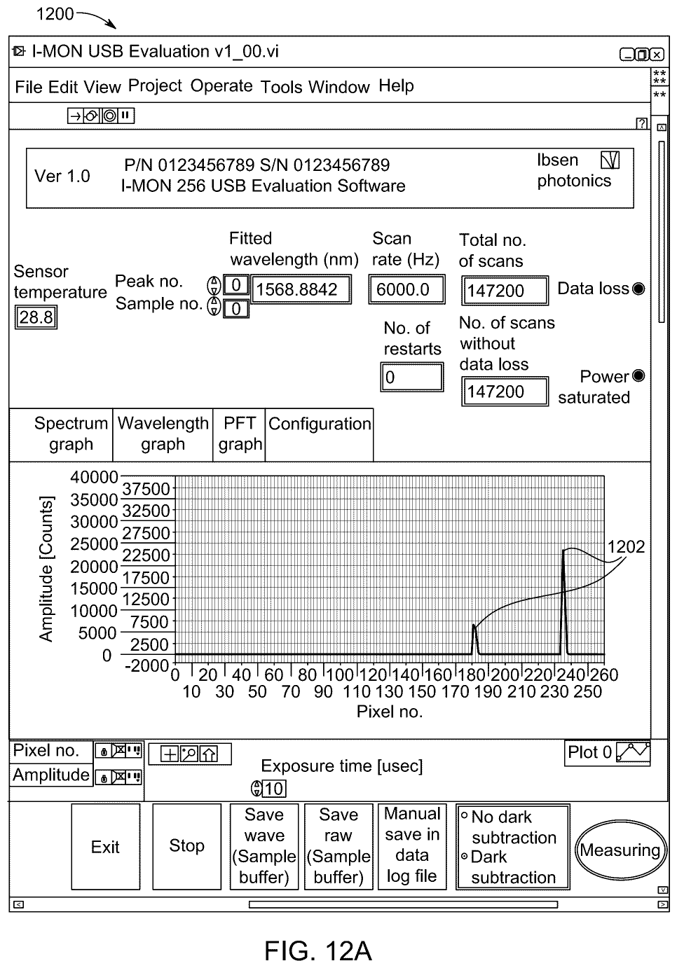

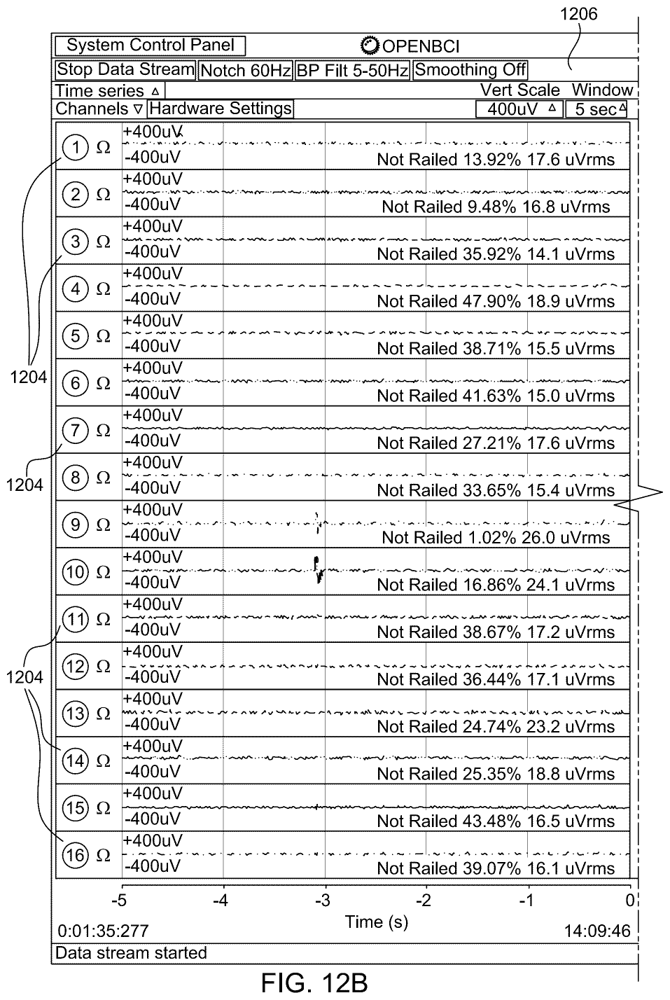

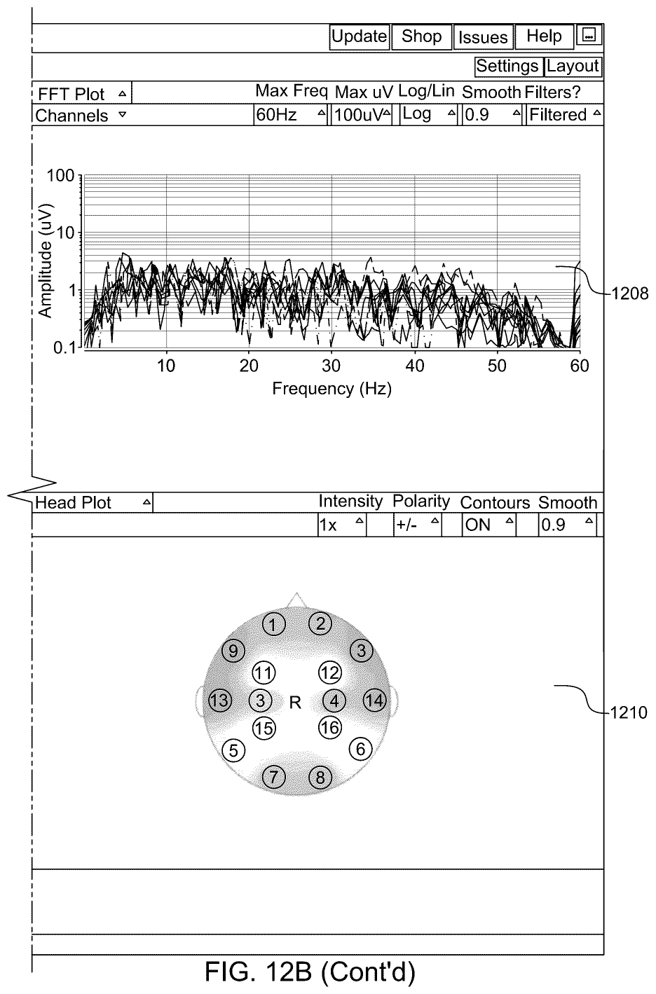

FIG. 12A illustrates an exemplary graphical user interface 1200 of the optical interrogator 1008, according to an embodiment. The reflected optical wavelength signal when received by the optical interrogator 1008, the display unit of the optical interrogator 1008 displays the reflected optical wavelength signal over the graphical user interface 1200. Spike Signals 1202 show the reflected wavelength signal, as an example. In an embodiment, if the number of FBG sensors are three, the number of spikes would be three in the graphical user interface 1200. Also, if all three FBG sensor 1006 undergo a plantar pressure due to applied pressure of the subject after wearing the wearable sandal arrangement 1014 on his foot, shift in wavelength corresponding to all three FBG sensor 1006 and corresponding to applied intensity of plantar pressure is simultaneously projected on the graphical user interface 1200 of the display unit. In such case, 3 spikes may be displayed with shift in wavelength compared to previous wavelength signal corresponding to each FBG sensor 1200 with no plantar pressure applied.

FIG. 12B illustrates an exemplary user interface 1206 illustrates an exemplary user interface illustrating a control panel for 16 channel EEG data collection. The user interface 1206 shows, as an example, the response or voltage pattern of 16 channels 1204 on the left side of the user interface 1206, when the subject wears the BCI 202 on his or her head and perform any specific state, such as sitting. Similarly, other voltage patterns are generated on 16 channels when the subject perform standing or walking. The amplitude values i.e., micro volt (μV) readings, are used as the data to interpret brain response to various foot movements during posture change and walking action. Further, the top right corner of the user interface 1206 shows Fourier transform (Amplitude vs. Frequency-FFT) voltage plot 1208 of the brain activity on all 16 channels. The bottom right corner of the user interface 1206 shows the head plot 1210 indicating activity region in the brain corresponding to various body actions of the user and 16 electrode positioning on the user head.

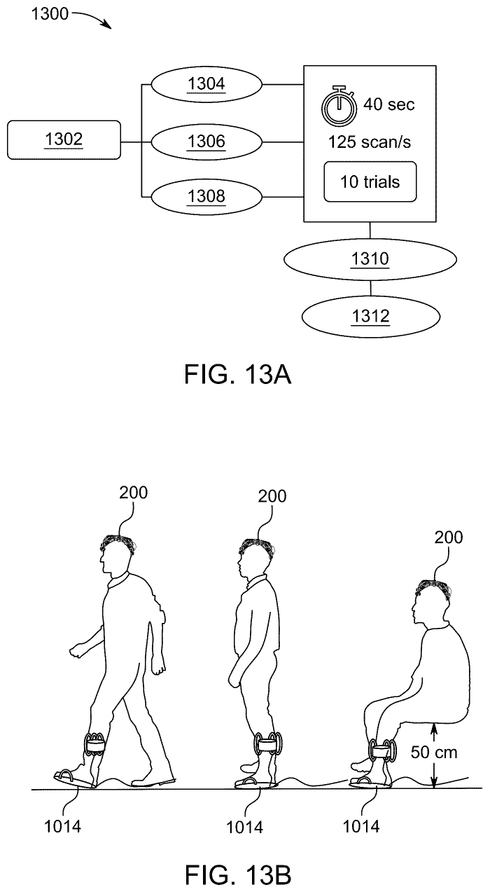

FIG. 13A illustrates the BCI prediction and the touch signal generation process 1300, according to an embodiment. The BCI prediction and the touch signal generation process 1300 is described in detail in conjugation with FIGS. 2, 3, 4, 5, 6, 7, 8, 9, 10, 11 and 12. The BCI prediction and the touch signal generation process 1300 includes processing of the second part of the experiment. A block 1302 shows a presence of 3 FBG sensors that is integrated into the wearable sandal arrangement 1014. The baseline wavelength signal of the first FBG sensor 1006-1, the second FBG sensor 1006-2 and the third FBG sensor 1006-3 are in a range of 1545 nm to 1550 nm, 1530 nm to 1535 nm, 1536 nm to 1540 nm, respectively. The baseline wavelength signal indicates that when a light signal is transmitted to the plurality of FBG 1006, the reflected signal would have wavelength in a range of 1545 nm to 1550 nm, 1530 nm to 1535 nm, 1536 nm to 1540 nm, respectively, that is registered by the optical interrogator 1008, when no plantar pressure is applied over the insole 1012, that is, the person has not worn the wearable sandal arrangement 1014. Also, the optical interrogator 1008 registers the peaks for each FBG sensor by its corresponding wavelength signature, that is, 1545.341 nm, 1535.068 nm, 1539.966 nm, respectively, as no plantar pressure applied on the wearable sandal arrangement 1014.

Now, initially, a subject wears the BCI 202 on his or her head, such that the 16 electrodes of the BCI 202 comes in contact with the head scalp 204. At the same time, the subject wears the wearable sandal arrangement 1014. Based on the result of the first part of the experiment, electrodes of the BCI 202 responding to maximum activity of the subject were identified as 2, 5, 6, 9, 11 and 12. The subject is now instructed to perform a sitting state having his or her foot on the ground. A block 1304 illustrates the sitting position of the subject during the experiment. Since the subject has worn the wearable sandal arrangement 1014 and being his or her foot on the ground, the plantar pressure due to foot of the subject is now applied on each FBG sensors 1006-1, 1006-2 and 1006-3. As such, applied plantar pressure is registered by the plurality of fiber bragg grating (FBG) sensors 1006.

The broadband light source 1002 illuminates, via the first port of the optical circulator 1004 and under the commend and/or control of computer controller 1010, the first FBG sensor 1006-1, the second FBG sensor 1006-2 and the third FBG sensor 1006-3. In response to the application of the broadband light source 1002, each of the plurality of FGB sensors 1006 outputs a unique wavelength shift due to the applied plantar pressure on the wearable sandal arrangement 1014 due to wavelength reflection property of the FBG sensor 1006. The each of the plurality of FBG sensors 1006 reflects a wavelength signal with a shift in wavelength due to applied plantar pressure on the wearable sandal arrangement 1014 in the sitting state. The reflected wavelength signal with a shift in wavelength travels through the optical circulator 1004 from the plurality of FBG sensors 1006 to the optical interrogator 1008. The optical interrogator 1008 being on the third port of the optical circulator 1004 receives the reflected wavelength signal.

Block 1310 shows the computation of wavelength shift in the reflected wavelength signal. In an embodiment, wavelength shifts of each of the plurality of FGB sensors 1006 do not overlap. For example, the FBG sensors 1006 are constructed in such way that the geometrical properties of each FBG sensor 1006 are unique. As such, the wavelength signal due to applied plantar pressure in one FBG sensor 1006, for example the first FBG sensor 1006-1, does not coincide with the wavelength signal due to applied plantar pressure in other FBG sensor 1006, for example, the second FBG sensor 1006-2.

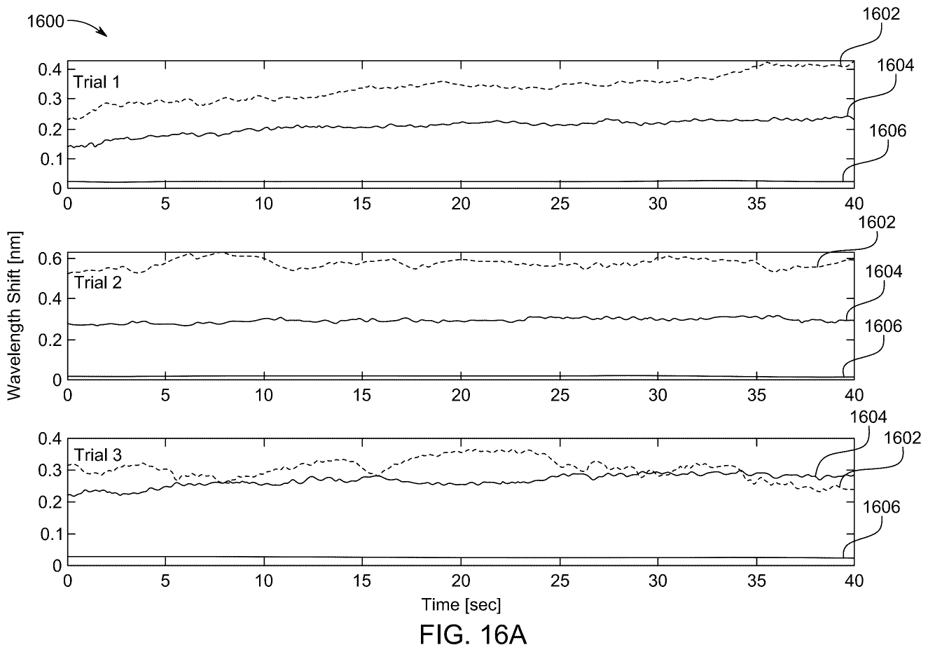

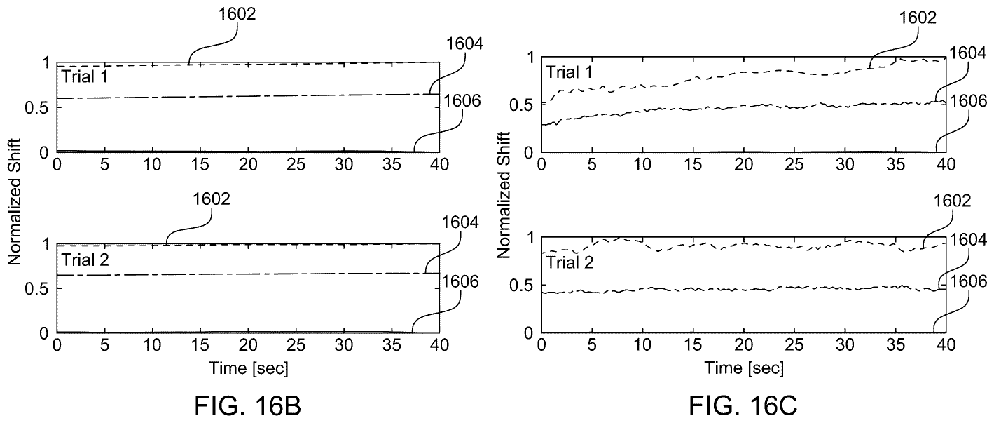

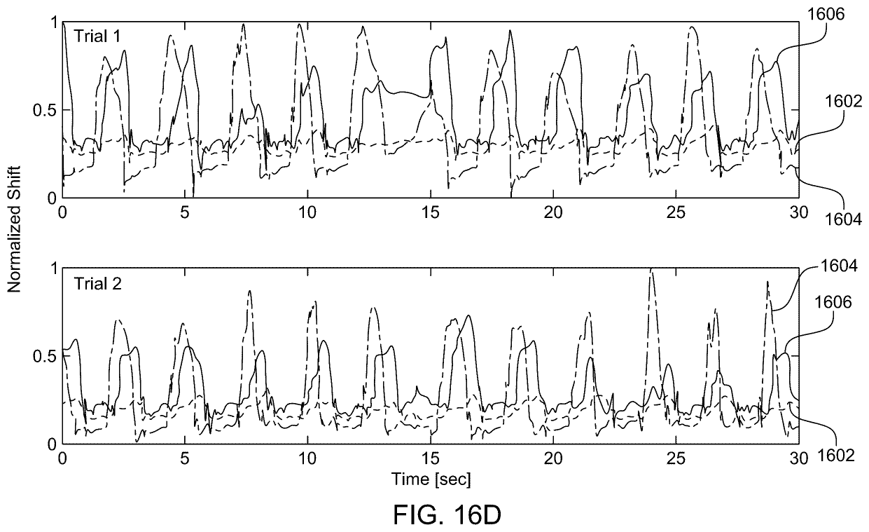

After receiving the reflected wavelength signal, the optical interrogator 1008 computes the shift in wavelength in each of plurality of FBG sensors 1006. For example, wavelength signal is calculated, via a processing circuitry of the optical interrogator 1008, as the difference between the base wavelength that is, 1545.341 nm, 1535.068 nm, 1539.966 nm and the wavelength shift. For example, the wavelength shift signal is calculated by the optical interrogator 1008 based on a difference between the baseline wavelength signal that is, 1545.341 nm, 1535.068 nm, 1539.966 nm and a peak wavelength signal. Mathematically, the wavelength shift is shown as:

$$Δ λ = {λ}_{orig} - {λ}_{meas} , \qquad ( 5 )$$

where, Δλ is wavelength shift, λ_orig is the original FBG wavelength or the baseline wavelength signal for the FBG sensor 1006 and λ_meas is the measured wavelength or the peak wavelength signal of the FBG sensor 1006. Also, the peak wavelength signal corresponds to a new wavelength signal due to applied plantar pressure of the foot of the subject. As an exemplary embodiment, the optical interrogator 1008 displays the wavelength shift data of the FBG sensor 1006 corresponding to sitting, standing and walking state on the user interface 1200 as shown in FIG. 12, for example. For example, FIG. 16A, illustrates an experimentally computed wavelength shift 1600 registered by plurality of FBG sensor 1006 in standing state, according to an embodiment. The optical interrogator 1008 shows a graph 1602 shows the wavelength shift registered by the second FBG sensor 1006-2 (middle region). A plot line 1604 shows the wavelength shift registered by the third FBG sensor 1006-3 (heel region). A plot line 1606 shows the wavelength shift registered by the first FBG sensor 1006-2 (toe region). Plurality of plot lines, that is, 1602, 1604 and 1606 are shown for the first trail in standing position for a single participant. Similarly, the optical interrogator 1008 also displays the plurality of plot lines 1612, 1614 and 1616 for first, second trial and the plurality of plot lines that is, 1622, 1624 and 1626 for third trial, respectively.

Referring back to FIG. 13A, block 1312 shows the normalization process of the computed shift in wavelength. Once the wavelength shift signal is calculated by the optical interrogator 1008, a normalization process is initiated. Since the computed value is affected by many factors, for example, participant's weight, FBG positions, surface material and specifications, and participant's posture. To minimize the error of a machine learning model that is used ahead in experiment while using the FBG data, all the collected data of the FBG sensor 1006 are further normalized using below equation:

$$Δ {λ}_{norm} = \frac{{λ}_{meas} - min ( of 3 waveforms )}{max ( of 3 waveforms ) - min ( of 3 waveforms )} \qquad ( 6 )$$

where, Δλ_norm is the normalized wavelength shift of the received wavelength signal.

After computing the normalized wavelength signal, the optical interrogator 1008 displays the wavelength shift corresponding to each of the plurality of FGB sensors 1006. For example, wavelength shift corresponding to the first FBG sensor 1006-1, the second FBG sensor 1006-2 and the third FBG sensor 1006-3 is displayed or projected on the display unit of the optical interrogator 1008, as shown in the graphical user interface 1200 of the optical interrogator 1008 in FIG. 12 corresponding to sitting state of the subject.

As an exemplary embodiment, the optical interrogator 1008 displays, after normalization process, the wavelength shift data of the FBG sensor 1006 corresponding to sitting, standing and walking state on the user interface 1200. For example, FIG. 16B illustrates an experimentally computed wavelength shift 1600 registered by plurality of FBG sensor 1006 in sitting state after normalization, according to an embodiment. The optical interrogator 1008 shows a plot line 1632 as the normalized wavelength shift registered by the second FBG sensor 1006-2 (middle region). A plot line 1634 shows a normalized wavelength shift registered by the third FBG sensor 1006-3 (heel region). A plot line 1636 shows a normalized wavelength shift registered by the first FBG sensor 1006-2 (toe region). Plurality of plot lines, that is, 1642, 1644 and 1646 are shown for the first trail in sitting position for a single participant. Similarly, the optical interrogator 1008 also displays the plurality of plot lines, that is, 1652, 1654 and 1656 for the second trial.

FIG. 16C illustrates an experimentally computed wavelength shift 1600 registered by plurality of FBG sensor 1006 in the standing state after normalization, according to an embodiment. The optical interrogator 1008 shows a graph 1662 as the normalized wavelength shift registered by the second FBG sensor 1006-2 (middle region). The plot line 1664 shows a normalized wavelength shift registered by the third FBG sensor 1006-3 (heel region). A plot 1666 shows a normalized wavelength shift registered by the first FBG sensor 1006-2 (toe region). Plurality of graphs that is, 1662, 1664 and 1666 are shown for the first trail in standing position for a single participant. Similarly, the optical interrogator 1008 also displays the plurality of graphs that is, 1672, 1674 and 1676 for the second trial.

FIG. 16D illustrates an experimentally computed wavelength shift registered by plurality of FBG sensor 1006 in walking state after normalization, according to an embodiment. The optical interrogator 1008 shows a graph 1682 as the normalized wavelength shift registered by the second FBG sensor 1006-2 (middle region). A graph 1684 shows a normalized wavelength shift registered by the third FBG sensor 1006-3 (heel region). A graph 1686 shows a normalized wavelength shift registered by the first FBG sensor 1006-2 (toe region). Plurality of graphs that is, 1682, 1684 and 1686 are shown for the first trail in standing position for a single participant. Similarly, the optical interrogator 1008 also displays the plurality of graphs that is, 1692, 1694 and 1696 for the second trial.

Referring back to FIG. 13A, after projecting the wavelength shift corresponding to the first FBG sensor 1006-1, the second FBG sensor 1006-2 and the third FBG sensor 1006-3 on the display unit of the optical interrogator 1008, the optical interrogator 1008 communications the data of the wavelength signal of the first FBG sensor 1006-1, the second FBG sensor 1006-2 and the third FBG sensor 1006-3. The computer controller 1010 receives the wavelength signals corresponding to the applied plantar pressure from the foot of the subject corresponding to sitting state of the subject.

The computer controller 1010 also receives the electroencephalography (EEG) signals using the BCI 202, corresponding to brain signals of the subject in the sitting state.

The step of receiving the wavelength signals corresponding to the applied plantar pressure from the foot of the subject corresponding to sitting state of the subject is repeated for at least 10 times by at least 10 different participants for at least in between 40-60 seconds. The EEG signals are received using the BCI 202, corresponding to brain signals of the subject in the sitting state is also repeated for at least 10 times by at least 10 different participants for at least in between 40-60 seconds.

Accordingly, the computer controller 1010 receives, with the processing circuitry of the computer controller 1010, a plurality of wavelength signals corresponding to the applied plantar pressure from the foot of the subject corresponding to the sitting state of the subject. Also, the computer controller 1010 receives, with the processing circuitry of the computer controller 1010, the plurality of electroencephalography (EEG) signals corresponding to brain signals of the subject corresponding to the sitting state of the subject.

The plurality of EEG signals is further transmitted to the classifier of the computer controller 1010. Also, plurality of wavelength signals of plurality of FBG sensor 1006 are also transmitted to the classifiers of the computer controller 1010. The classifier of the computer controller thus performs a correlation between the plurality of EEG signals and the plurality of wavelength signals. The correlation between the plurality of EEG signals and the plurality of wavelength signals helps the classifier to understand the pattern of the EEG signals at the sitting state as well as identifying the electrodes or channels of the BCI 202 showing maximum response corresponding to sitting state.

Since in the first part of the experiment, the identified channels were 2, 5, 6, 9, 11 and 12 that shows maximum response to the state of the subject state whether the subject is in sitting state, standing state or in the walking state. Also, during the second part of the experiment, the classifier may again find the correlation that at which electrodes or channels, the maximum response of brain signals are detected. Accordingly the classifier selects only a subsection of the channels that is, channels 2, 5, 6, 9, 11 and 12, confirmed from the first part of the experiment using KNN or Naïve bayes classifier or by performing the correlation in between the EEG signals and the wavelength signal of FBG sensors 1006 in the second part of the experiment.

In an embodiment, the number of channels 2, 5, 6, 9, 11 and 12 identified to show maximum response to the state of the subject is considered as an example only. The classifier may again, based upon the brain activity found on plurality of channels over plurality of subjects, identify the number of channels that show maximum response of the brain activity in plurality of states of the subject. In an example, the different channels other than channels 2, 5, 6, 9, 11 and 12 may be identified such as channels and is considered as a subsection of channels.

Further, the processing circuitry of the computer controller 1010, after forming the secondary subset of the channels that shows maximum response of brain signals, combines the voltage data available over these subset of channels that is, over channels 2, 5, 6, 9, 11 and 12 and the wavelength signal data of the FBG sensor 1006. The combination of the EEG signal data on the subset of the channels along with wavelength signal of the FBG sensor 1006 forms a secondary dataset. The secondary dataset thus includes a pattern of the brain signals or the voltage signals and corresponding wavelength signals of the FBG sensor 1006 for multiple trials for multiple number of participants. The secondary dataset corresponding to sitting state is further passed to a machine learning model of the computer controller 1010.

In an embodiment, the process including receiving the wavelength signals corresponding to the applied plantar pressure from the foot of the subject corresponding to sitting state of the subject is repeated again at least 10 times by at least 10 different participants for at least in between 40-60 seconds. The step of receiving the EEG signals using the BCI 202, corresponding to brain signals of the subject in the sitting state is also repeated for at least 10 times by at least 10 different participants for at least in between 40-60 seconds.

In an embodiment, the secondary data set includes EEG signal data as well as FBG data, and the formation of the secondary data set is described as follows. The dataset consisting of several sitting data points for BCI channels, for example 84 standing data points. The processor of the computing device performs the wavelet decomposition of the sitting data of the BCI signals to remove the noise signal. The processor of the computing controller 1010 simultaneously preserves the raw EEG signal data, wavelet data and the processed data/normalized data of EEG signal data. Similarly, for FBG data corresponding to sitting state, the processor of the computing controller 1010 normalizes the FBG datafiles by finding the maximum and minimum points of the FBG datafiles in standing position. In an embodiment, the normalization is performed to change the values of numeric columns in the dataset to a common scale without distorting differences in the ranges of values. Also, the FGB data is normalized to [0, 1] using min-max scaling. Further, the processor of the computing controller 1010 combines the FBG dataset and the raw, wavelet and processed/normalized dataset of EEG signal data into one file, as a secondary dataset with more than 1750 sample of BCI and the FBG dataset. The secondary dataset corresponding to sitting state of the subject is now ready to be provided into the machine learning model.

The subject is instructed to move to a standing state for collecting the EEG signal data as well as FBG data of the subject corresponding to standing state and form a secondary subset of the EEG signal data and the FBG sensor data to train the machine learning model. A block 1306 shows a standing position of the subject during the experiment. Since in standing position, the plantar pressure is increased, and accordingly, the shift in wavelength signal in the first FBG sensor 1006-1, the second FBG sensor 1006-2 and the third FBG sensor 1006-3 may be observed. Similar procedure is again repeated as described earlier in case of sitting position, that is summarized herein and is not discussed in detail. The repeated process in case of standing condition includes multiple processes such as computation of shift in wavelength of the reflected signal due to plantar pressure corresponding to standing position, normalization process and displaying the wavelength shift corresponding to each of the plurality of FGB sensors 1006. The computer controller 1010 further receives, with the processing circuitry of the computer controller 1010, a plurality of wavelength signals corresponding to the applied plantar pressure from the foot of the subject corresponding to the standing state of the subject. Also, the computer controller 1010 receives, with the processing circuitry of the computer controller 1010, the plurality of electroencephalography (EEG) signals corresponding to brain signals of the subject corresponding to the standing state of the subject, followed by correlation process between the EEG signals and the wavelength signal of FBG sensors 1006 to form a secondary dataset of multiple trails and multiple participants, followed by passing the secondary dataset corresponding to standing state to the machine learning model of the computer controller 1010.

In an embodiment, the process, that is, receiving the wavelength signals corresponding to the applied plantar pressure from the foot of the subject corresponding to the standing state of the subject is again repeated at least 10 times by at least 10 different participants for at least in between 40-60 seconds. The process including receiving the electroencephalography (EEG) signals using the BCI 202, corresponding to brain signals of the subject in the standing state is also repeated at least 10 times by at least 10 different participants for at least in between 40-60 seconds.

In an embodiment, the secondary data set includes EEG signal data as well as FBG data is described as follows. The dataset including several standing data for BCI channels, for example, 102 standing data. The processor of the computing device perform the wavelet decomposition of the standing data of the BCI signals to remove the noise signal. The processor of the computing controller 1010 simultaneously preserves the raw EEG signal data, wavelet data and the processed data/normalized data of EEG signal data. Similarly, for FBG data corresponding to standing state, the processor of the computing controller 1010 normalizes the FBG datafiles by finding the maximum and minimum points of the FBG datafiles in standing position. In an embodiment, the normalization is performed to change the values of numeric columns in the dataset to a common scale without distorting differences in the ranges of values. Also, the FGB data is normalized to [0, 1] using min-max scaling. Further, the processor of the computing controller 1010 combines the FBG dataset and the raw, wavelet and processed/normalized dataset of EEG signal data into one file, as a secondary dataset with more than 1750 sample of BCI and the FBG dataset. The secondary dataset corresponding to standing state of the subject is now ready to be provided into the machine learning model.

The subject is instructed to transition to the walking state for collecting the EEG signal data as well as FBG data of the subject corresponding to the walking state and form a secondary subset of the EEG signal data and the FBG sensor data to train the machine learning model. Accordingly, a block 1308 shows a walking position of the subject during the experiment. In the walking position, the plantar pressure on each FBG sensor 1006 is again modified. Accordingly, the shift in wavelength signal in the first FBG sensor 1006-1, the second FBG sensor 1006-2 and the third FBG sensor 1006-3 may again be observed. The process is again repeated as described earlier in case of the sitting state and the standing state, that is summarized herein and is not discussed in detail. The repeated process in the case of the walking state includes multiple processes such as computation of shift in wavelength of the reflected signal due to plantar pressure corresponding to walking state, normalization process and displaying the wavelength shift corresponding to each of the plurality of FGB sensors 1006. The computer controller 1010 further receives, with the processing circuitry of the computer controller 1010, a plurality of wavelength signals corresponding to the applied plantar pressure from the foot of the subject corresponding to the walking position of the subject. Also, the computer controller 1010 receives, with the processing circuitry of the computer controller 1010, the plurality of EEG signals corresponding to brain signals of the subject corresponding to the walking state of the subject, followed by correlation process between the EEG signals and the wavelength signal of FBG sensors 1006 to form a secondary dataset of multiple trails and multiple participants, followed by passing the secondary dataset corresponding to walking position to the machine learning model of the computer controller 1010.

In an embodiment, the process that is, receiving the wavelength signals corresponding to the applied plantar pressure from the foot of the subject corresponding to the walking state of the subject is again repeated for at least 10 times by at least 10 different participants for at least in between 40-60 seconds. The step of receiving the EEG signals using the BCI 202, corresponding to brain signals of the subject in walking state is also repeated at least 10 times by at least 10 different participants for at least in between 40-60 seconds.

In an embodiment, the secondary data set includes EEG signal data as well as FBG data, and is described as follows. The dataset consists of several walking data for BCI channels, for example 60 walking data. The processor of the computing controller 1010 performs the wavelet decomposition of the walking data of the BCI signals to remove the noise signal. The processor of the computing controller 1010 simultaneously preserves the raw EEG signal data, wavelet data and the processed data/normalized data of EEG signal data. Similarly, for FBG data corresponding to walking state, the processor of the computing controller 1010 normalizes the FBG datafiles by finding the maximum and minimum points of the FBG datafiles in walking position. In an embodiment, the normalization is performed to change the values of numeric columns in the dataset to a common scale without distorting differences in the ranges of values. Also, the FGB data is normalized to [0, 1] using min-max scaling. Further, the processor of the computing controller 1010 combines the FBG dataset and the raw, wavelet and processed/normalized dataset of EEG signal data into one file, as a secondary dataset with more than 1750 sample of BCI and the FBG dataset. The secondary dataset corresponding to walking state of the subject is now ready to be provided into the machine learning model.

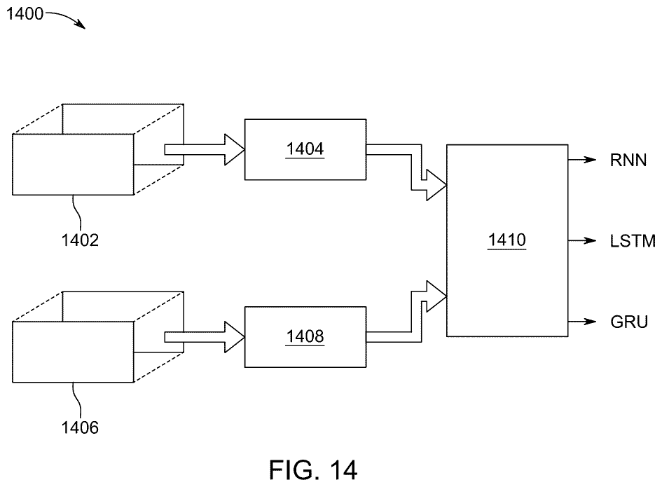

FIG. 13B illustrates the walking state, the standing state and the sitting state of the subjects while wearing the wearable sandal arrangement 1014 and the BCI 202, according to an embodiment. The walking data is recorded for 15 seconds, repeated 20 times for each of the participants for a total of 60 files, where the path allows for an average of 14 steps. The subjects are instructed to transition to a standing state without making changes in body posture other than standing posture. The standing data is collected for 60 seconds, repeated 10 to 15 times for a total of 51 files. The sitting data is collected for 60 seconds for each of the participants, each repeated to 12 times for a total of 43 sitting data files. The machine learning process for predicting brain signal and generating a touch signal is described in detail, based on the secondary dataset for sitting, standing and walking sate of the subject in FIG. 14.

FIG. 14 illustrates a signal flow diagram 1400 for machine learning process for predicting the brain signal and generating the touch signal, according to an embodiment. FIG. 14 is described in conjunction with FIG. 13A. A block 1402 shows collection of the EEG signal data from 16 channels using BCI 202 in plurality of states of subjects or participants. The collected EEG signal data is preprocessed in a block 1404, for example, raw data, normalization or wavelet transformation of the EEG signal data for removing noise, as explained earlier in FIG. 13. Similarly, a block 1406 shows collection of the data of FBG sensors 1006 from plurality of participants in plurality of states of subjects or participants. The collected FBG data is preprocessed in a block 1408, for example, normalization by finding the maximum and minimum points in the FBG dataset waveform. The computer controller 1010 device combines the EEG signal data and the FBG data, and provides the combined data to a block 1410 where both data are processed for providing to machine learning models. In an embodiment, the block 1404, the block 1408 and the block 1410 are part of the computing controller 1010 where the combined data is processed to be provided to the machine learning models.

The processor of the computing controller 1010 creates deep learning algorithms for brain activity electrodes corresponding to plurality of secondary datasets that is, the sitting state, the standing state and the walking state. For example, the computing controller 1010 uses three different models that is, RNN, LSTM and GRU models which is now described in detail. The RNN model and learning brain signals corresponding to sitting pattern of the subject is described first. The processor of the computer controller 1010 receives and provides the secondary dataset that includes raw data, processed/normalized data and the wavelet data of sitting pattern to the RNN model. The RNN model learns various data pattern related to FBG sensor 1006 and correlates with the data pattern of brain signals for the participants of sitting data. The RNN model keeps learning the various data of the FBG sensor 1006 that corresponds to the sitting activity of the participant that is generating the specific brain signal. For example, if a subject sits with his leg on the ground, the force on all three sensors may be small and so the wavelength shift pattern of the FBG data may also be small. Accordingly, the brain signals are generated corresponding to the subject's sitting state. The RNN model learns that when the wavelength shift in all the FBG sensor 1006 are small, it corresponds to a sitting pattern of the subject. In an example, the RNN model may learn the range of the shift of the wavelength of FBG signals that is generating the specific brain signal corresponds to the sitting position of the subject. Based on the learning process, the RNN model may predict, after few training samples of the EEG signal data and the FBG data, the brain signal corresponding to the any FBG dataset that corresponds to the sitting pattern of the subject or participants.

Similarly, the processor of the computer controller 1010 provides the secondary dataset that includes raw data, processed/normalized data and the wavelet data of standing pattern to the RNN model. The RNN model learns various data pattern related to FBG sensor 1006 and correlates with the data pattern of brain signals for plurality of participants of standing data. The RNN model keeps learning the various data of FBG sensor 1006 that corresponds to the standing activity of the participant that is generating the specific brain signal. For example, if the subject or participant stands, the force on all three FBG sensors 1006 is high compared to the force on the FBG signals 1006 when the participant is just sitting. Accordingly, the wavelength shift pattern of the FBG data is also high compared to the sitting position. Accordingly, the brain signals are generated due to the standing state. The RNN model learns that when the wavelength shift in all the FBG sensor 1006 are high, it may correspond to the standing pattern of the subject. In an example, the RNN model may learn the range of the shift of the wavelength of FBG signals that is generating the specific brain signal corresponding to the standing position of the subject. Based on the learning process, the RNN model may predict, after few training samples of the EEG signal data and the FBG data, the brain signal corresponding to the FBG dataset that corresponds to the standing pattern of the subject.

The processing circuitry of the computer controller 1010 provides the secondary dataset that includes raw data, processed/normalized data and the wavelet data of walking state to RNN model. The RNN model learns various data pattern related to FBG sensor 1006 and correlates with the data pattern of brain signals for plurality of participants on walking data. The RNN model keeps learning what are the various data of the FBG sensor 1006 that corresponds to the walking activity of participants that is generating the specific brain signal. For example, if a subject walks, the force on all three sensors may shows a monotonic increasing and decreasing pattern of pressure on all three FBG sensors 1006. As the gait of the subject changes or as the subject proceeds further, the pressure on the heel area reduces and the pressure on the mid area of the foot increases while the toe area may remain at the same pressure. Again as the person moves further, the weight of the subject's body comes mostly over the toe area thereby increasing a pressure in the toe area compared to the heel and mid portion of the foot. Accordingly, the wavelength shift pattern of the FBG data on the first FBG sensor 1006-1, the second FBG sensor 1006-2 and the third FBG sensor 1006-3 may also show a monotonic increase and decrease in shift in wavelength signal. Accordingly, the brain signals are generated due to walking state of the subject or the participant. The RNN model learns the monotonic increase and decrease in wavelength shift in FBG sensor data and tries to correlate the brain signal that is generating in the brain at the time of walking, that correspond to a walking pattern of the subject or participant. In an example, the RNN model may learn the range of the monotonic shift of the wavelength of FBG signals that is generating the specific brain signal corresponds to the walking position of the subject. Based on the learning process, the RNN model may predict, after few training samples, the brain signal corresponding to the any FBG dataset that corresponds to the walking pattern of the subject.

In an embodiment, to optimize each model, the processor of the computer controller 1010 uses a loss function. For example, the processor of the computer controller 1010 uses a mean square error (MSE) function to maximize the prediction accuracy of the RRN model. The MSE function is given by:

MSE=1/nΣᵢ₌₁ⁿ(yᵢ−ŷᵢ)², (7)

where n is the number of samples, yᵢ is the targeted value, and ŷᵢ is the predicted value.

Aforementioned description is provided for training the RNN model for predicting brain signals along with maximizing the prediction accuracy using the loss function. Similar process is again repeated for LSTM and GRU machine learning models and is not described or repeated here.

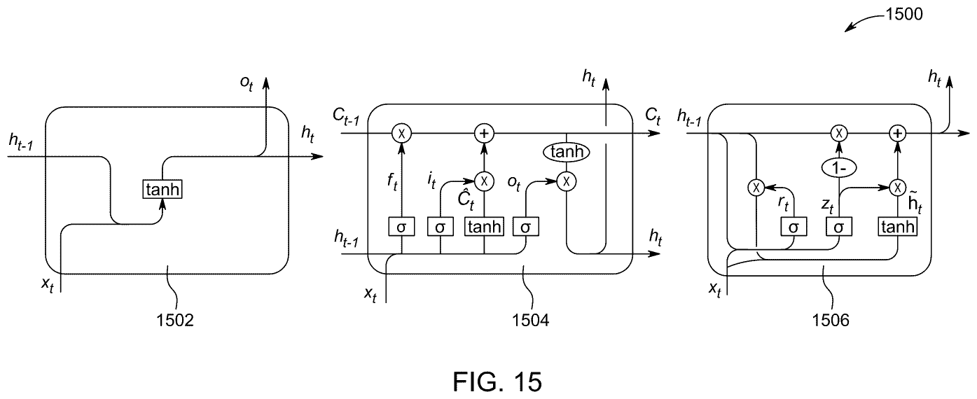

FIG. 15 illustrates a block diagram of each machine learning model, according to an embodiments. A block 1502 illustrates a block diagram of the RNN model. A block 1504 illustrates a block diagram of the LSTM model and a block 1506 illustrates a block diagram of the GRU model. Accordingly, all three machine learning models are trained using the secondary dataset of EEG signal data and FBG data. Once all models are trained to identify brain signals corresponding to state of the subject or participants, each machine learning models may undergo a testing phase to identify the BCI prediction accuracy of each machine learning model.

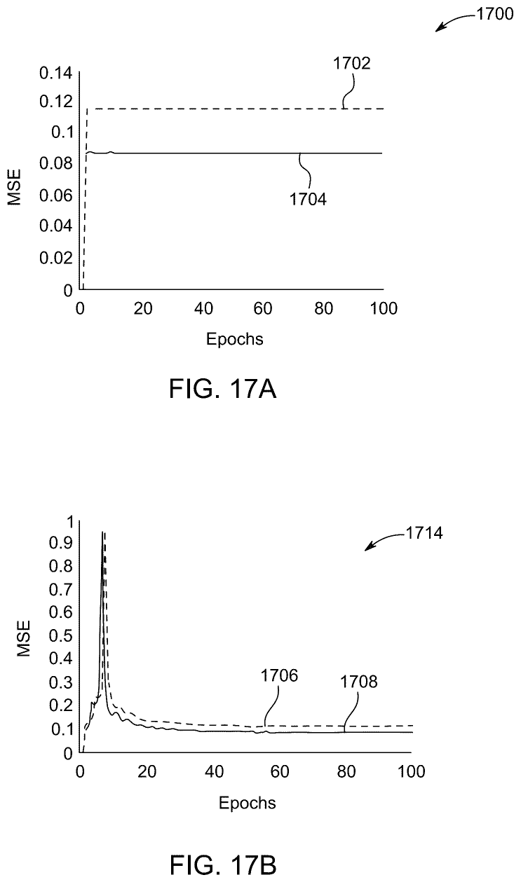

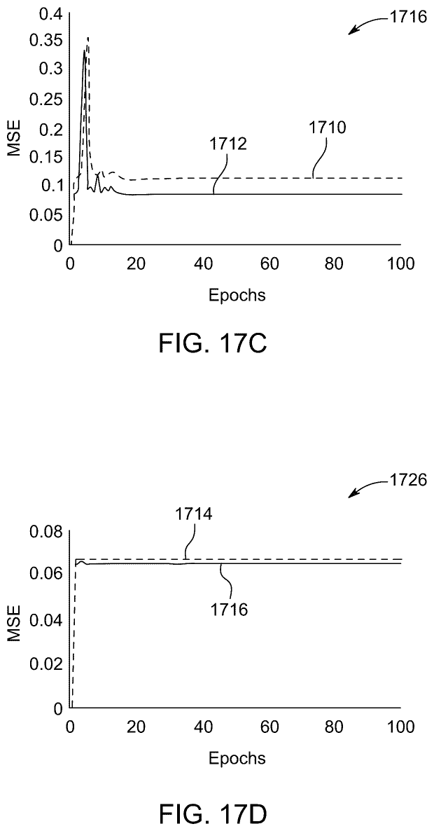

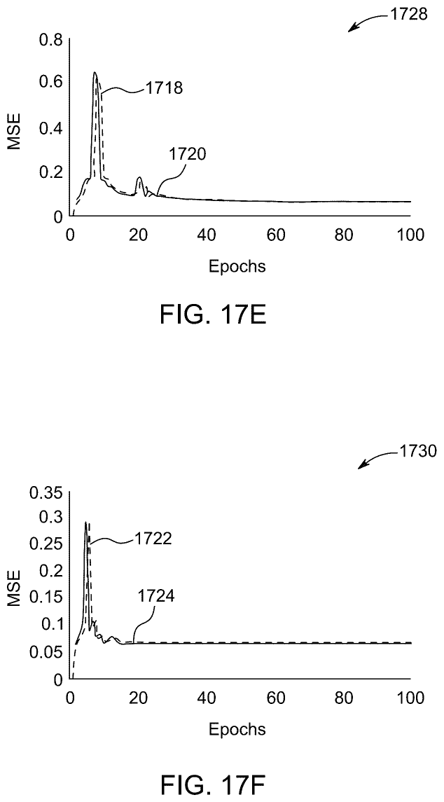

In the testing phase, 20% of the secondary dataset in the sitting state, the standing state and the walking state is used to test the BCI prediction accuracy of each model. For example, a subject wears the BCI 202 and the wearable sandal arrangement 1014. The subject is in the sitting state, the standing state and the walking state. The FBG sensor 1006 data is provided to the first machine learning model that is, RNN model in the sitting state, standing state and walking state. The RNN model predicts the EEG signal data, based upon the FBG data pattern, over plurality of channels that is, 2, 5, 6, 9, 11 and 12. The actual EEG signal data (brain signal) is also simultaneously detected on the same channel. The same process of training and testing is again repeated for the LSTM model and the GRU model, as described for RNN model. A mean square error (MSE) is computed based upon the predicted EEG signal data and the actual EEG signal data in each machine learning model. The MSE is plot for comparison to identify the accuracy of the models, as described in detail in FIG. 17.

FIG. 17A illustrates an exemplary plot 1700 of the values of the training and validation MSE of LSTM machine learning model for the processed data on channel 6, according to an embodiment. It was observed during the experiment that the raw data was not suitable for any machine learning model. A graph 1702 shows the training set of the mean square error (MSE) whereas a graph 1704 shows the testing phase of the MSE. It was observed that the LSTM has the least error and requires the least number of epochs to reach a steady state. As such the prediction accuracy of BCI signals of LSTM machine learning model was found to be better than other used machine learning models.

FIG. 17B illustrates an exemplary plot 1714 of the values of the training and validation MSE of RNN machine learning model for the processed data on channel 6, according to an embodiment. A graph 1706 shows training set of the mean square error (MSE) whereas a graph 1708 shows the testing phase of the MSE. It was found that the steady state is reached after a significant delay in case of RNN machine learning model.

FIG. 17C illustrates an exemplary plot 1716 of the values of the training and validation MSE of GRU machine learning model for the processed data on channel 6, according to an embodiment. A graph 1710 shows a training set of the mean square error (MSE) whereas a graph 1712 shows the testing phase of the MSE. It was found that the steady state is reached after a significant delay in case of GRU machine learning model.

FIG. 17D illustrates an exemplary plot 1726 of the values of the training and validation MSE of LSTM machine learning model for wavelet decomposition data on channel 9, according to an embodiment. A graph 1714 shows a training set of the mean square error (MSE) whereas a graph 1716 shows the testing phase of the MSE. It was observed that the LSTM has the least error and requires the least number of epochs to reach a steady state.

FIG. 17E illustrates an exemplary plot 1728 of the values of the training and validation MSE of RNN machine learning model for wavelet decomposition data on channel 9, according to an embodiment. A graph 1718 shows a training set of the mean square error (MSE) whereas a graph 1720 shows the testing phase of the MSE. It was found that the steady state is reached after a significant delay in case of RNN machine learning model.

FIG. 17F illustrates an exemplary plot 1730 of the values of the training and validation MSE of GRU machine learning model for wavelet decomposition data on channel 9, according to an embodiment. A graph 1722 shows a training set of the mean square error (MSE) whereas a graph 1724 shows the testing phase of the MSE. It was found that the steady state is reached after a significant delay in case of the GRU machine learning model.

Considering the result of FIG. 17A-FIG. 17F, it was identified that the performance of LSTM machine learning model performed better in predicting the BCI signals at plurality of BCI channels that is, 2, 5, 6, 9, 11 and 12, when the used EEG signal data are processed/normalized data or decomposition data.

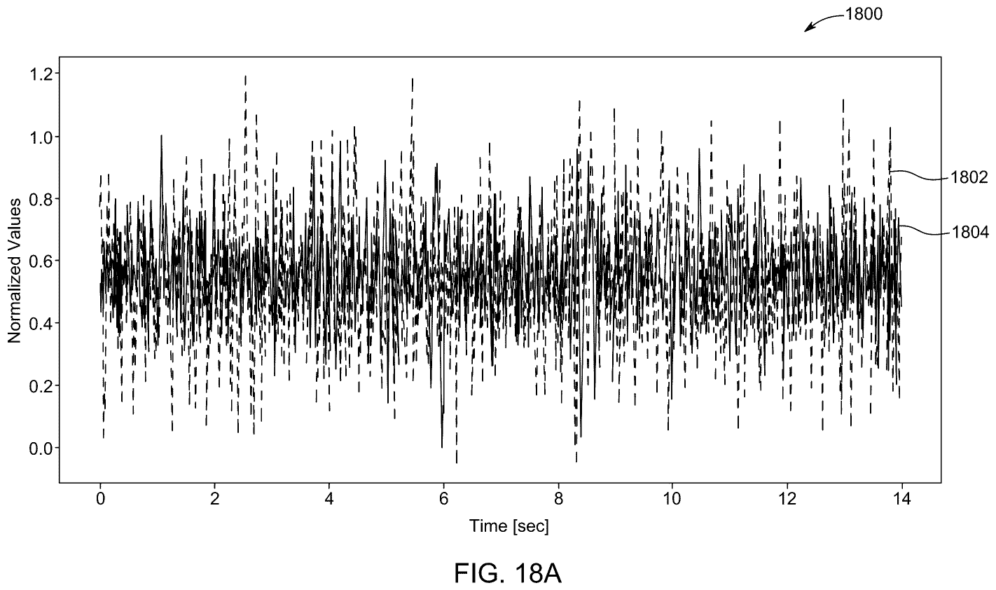

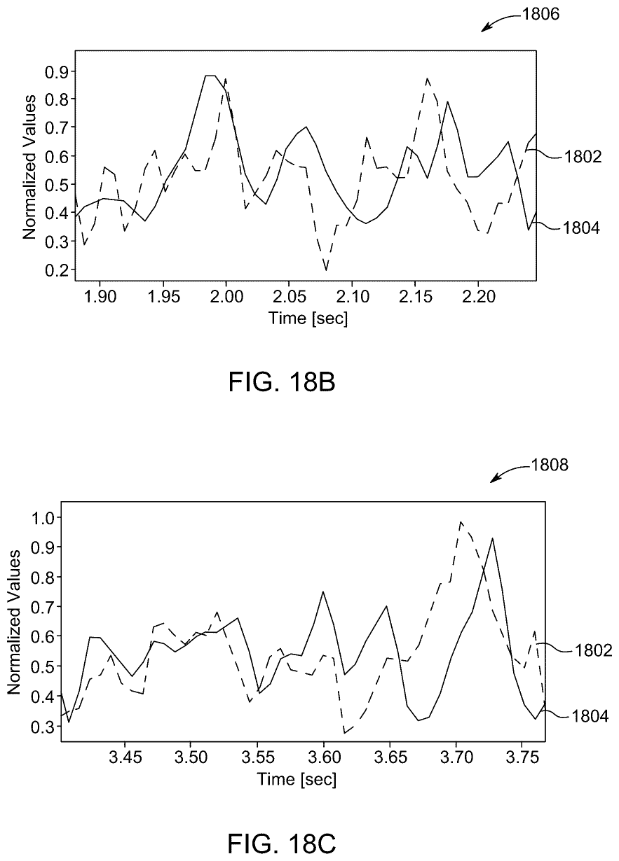

FIG. 18A illustrates a comparison graph 1800 between the original and predicted EEG signal data at channel 2 for an unknown FBG sensor data, according to an embodiment. It was found in FIG. 17 that the LSTM model performed better compared to the other two models. Therefore, an unknown pattern of FBG sensor data is provided to the LSTM model. A plot line 1802 shows a predicted EEG signal data whereas a plot line 1804 shows the original EEG signal data.

FIG. 18B illustrates a portion of graph 1800 that is magnified from 1.90 seconds to 2.25 seconds. A plot line 1812 shows a predicted EEG data, whereas a plot line 1814 shows the original EEG data.

FIG. 18C illustrates a portion of graph 1800 that is magnified from 3.4 seconds to 3.8 seconds. A plot line 1822 shows a predicted EEG data whereas a plot line 1824 shows the original EEG data.

Based upon the result of FIG. 18A-FIG. 18C, the predicted EEG data on any arbitrary FBG sensor data, the LSTM machine learning model provides a good results and therefore close resemblance is identified between the original and the predicted BCI signal.

In an embodiment, the touch signal is transmitted from the artificial lower limb prosthesis to a haptic feedback system. The haptic feedback system may include a wearable system that could be worn on the body of the amputee such as a hand, finger, chest, neck or the like. In an embodiment, the hepatic wearable system 222 may include a vest worn on the subject's chest. The haptic feedback system 222 may be configured to perform a wired or wireless communication with the prosthetic device. For example, once the touch signal is relayed to the artificial lower limb prosthesis, the artificial lower limb prosthesis may communicate the touch signal to the vest. Based on the generated touch signal, that is, sitting state, standing state or the walking state, the vest may be configured to convert the touch signal into, for example, a vibration or gripping force to the body of the amputee, that is applied to the vest of the subject. For example, if the generated touch signal is due to sitting state of the amputee, the intensity of vibration or gripping force at the vest of the amputee may be low. Similarly, if the generated touch signal is due to standing state of the amputee, the intensity of vibration or gripping force at the vest of the amputee may be a bit high compared to the vibration intension at sitting state. Also, if the generated touch signal is due to walking state of the amputee, the intensity of vibration or gripping force at the vest of the amputee may be a high compared to the vibration intension at standing state. In another example, when an amputee is in walking state, the haptic feedback system may receive a monotonic increase in vibration.

In another embodiment, a frequency of vibration may be based upon the state of the amputee. For example, if the generated touch signal is due to sitting state of the amputee, the range of the frequency of vibration at the vest of the amputee may be small, for example, 100-200 Hz may be used. Similarly, if the generated touch signal is due to standing state of the amputee, the range of the frequency of vibration at the vest of the amputee may be a bit high, for example 1000-2000 Hz may be used. Also, if the generated touch signal is due to walking state of the amputee, the range of the frequency of vibration at the vest of the amputee may be 3000-4000 Hz. Accordingly, the touch signal elicits a haptic response corresponding to the subject's state. As such, there may be plurality of ways to relay the touch signal to the haptic feedback system that aids the touch sensation to the body of the amputee due to either sitting state, standing state or the walking state.

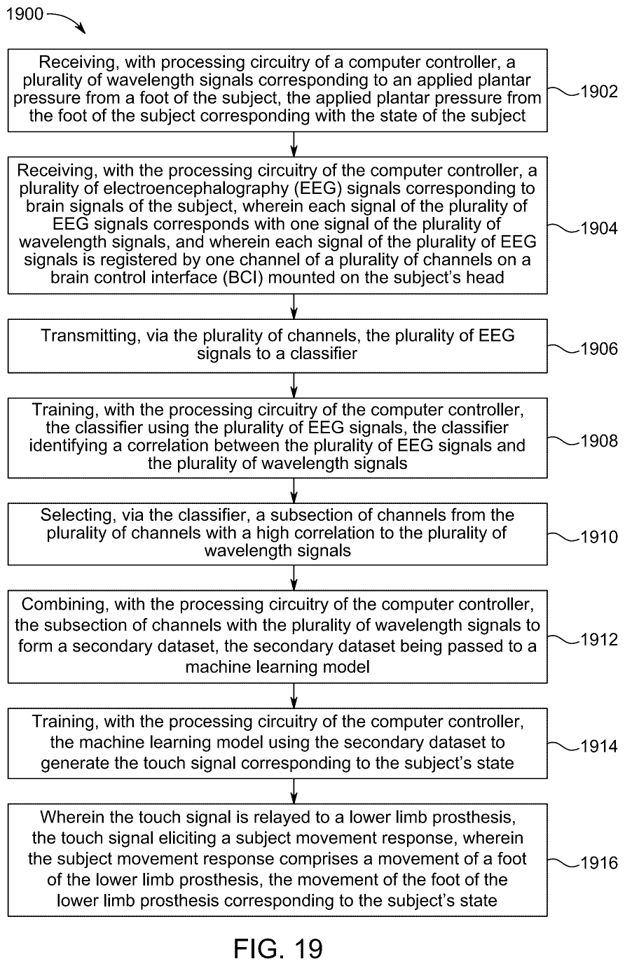

FIG. 19 illustrates a flowchart of a method 1900 of generating a touch signal corresponding to a state of a subject, according to an embodiment. Various steps of the method 1900 are included through blocks in FIG. 19. One or more blocks may be combined or eliminated to achieve the objective of methods for generating a touch signal corresponding to a state of a subject without departing from the scope of the present disclosure.

At step 1902, the method 1900 includes receiving, with processing circuitry of a computer controller 1010, a plurality of wavelength signals corresponding to an applied plantar pressure from a foot of the subject. The applied plantar pressure from the foot of the subject corresponds with the state of the subject.

At step 1904, the method 1900 further includes receiving, with the processing circuitry of the computer controller 1080, a plurality of EEG signals corresponding to brain signals of the subject. Each signal of the plurality of EEG signals corresponds with one signal of the plurality of wavelength signals. Each signal of the plurality of EEG signals is registered by one channel of a plurality of channels on a brain control interface (BCI) 200 mounted on the subject's head, At step 1906, the method 1900 further includes transmitting, via the plurality of channels, the plurality of EEG signals to a classifier. In an aspect, the classifier is an integral part of the computer controller 1080.

At step 1908, the method 1900 further includes training, with the processing circuitry of the computer controller 1080, the classifier using the plurality of EEG signals, the classifier identifying a correlation between the plurality of EEG signals and the plurality of wavelength signals.

At step 1910, the method 1900 further includes selecting, via the classifier, a subsection of channels from the plurality of channels with a high correlation to the plurality of wavelength signals.

At step 1912, the method 1900 further includes combining, with the processing circuitry of the computer controller 1080, the subsection of channels with the plurality of wavelength signals to form a secondary dataset, the secondary dataset being passed to a machine learning model.

At step 1914, the method 1900 further includes training, with the processing circuitry of the computer controller, the machine learning model using the secondary dataset to generate the touch signal corresponding to the subject's state.

At step 1916, the method 1900 further includes relaying the touch signal to a lower limb prosthesis. The touch signal elicits a subject movement response. The subject movement response comprises a movement of a foot of the lower limb prosthesis. The movement of the foot of the lower limb prosthesis corresponding to the subject's state.

Numerous modifications and variations of the present disclosure are possible in light of the above teachings. For example, more than three FBG sensor may be placed in the insole of the sandal arrangement for increasing the accuracy in computing the plantar pressure and hence the brain signals. In place of the FBG sensor, other pressure-based sensor known in the art may be used. Also, the invention discloses about the plantar pressure measurement and generating the brain signal based upon the plantar pressure measurement. However, in case a subject who has lost his hand, the invention may again be modified and used for generating the brain signal based on subject's palm pressure on any surface, based upon the state of the palm. Therefore, there are plenty of possible modification that can be introduced while describing or practicing the invention. It is therefore to be understood that within the scope of the appended claims, the invention may be practiced otherwise than as specifically described herein.

1. A method of generating a touch signal corresponding to a state of a subject comprising:
receiving, with processing circuitry of a computer controller, a plurality of wavelength signals corresponding to an applied plantar pressure from a foot of the subject, the applied plantar pressure from the foot of the subject corresponding with the state of the subject,
receiving, with the processing circuitry of the computer controller, a plurality of electroencephalography (EEG) signals corresponding to brain signals of the subject,
wherein each signal of the plurality of EEG signals corresponds with one signal of the plurality of wavelength signals, and wherein each signal of the plurality of EEG signals is registered by one channel of a plurality of channels on a brain control interface (BCI) mounted on the subject's head,
transmitting, via the plurality of channels, the plurality of EEG signals to a classifier,
training, with the processing circuitry of the computer controller, the classifier using the plurality of EEG signals, the classifier identifying a correlation between the plurality of EEG signals and the plurality of wavelength signals,
selecting, via the classifier, a subsection of channels from the plurality of channels with a high correlation to the plurality of wavelength signals,
combining, with the processing circuitry of the computer controller, the subsection of channels with the plurality of wavelength signals to form a secondary dataset, the secondary dataset being passed to a machine learning model,
training, with the processing circuitry of the computer controller, the machine learning model using the secondary dataset to generate the touch signal corresponding to the subject's state,
wherein the touch signal is relayed to a lower limb prosthesis,
the touch signal eliciting a subject movement response,
wherein the subject movement response comprises a movement of a foot of the lower limb prosthesis,
the movement of the foot of the lower limb prosthesis corresponding to the subject's state.

2. The method of claim 1, wherein the touch signal is transmitted from the lower limb prosthesis to a haptic feedback system,
the haptic feedback system comprising a vest worn on the subject's chest,
wherein the touch signal elicits a haptic response corresponding to the subject's state.

3. The method of claim 1, wherein the plurality of channels comprises 16 channels,
wherein the subsection of channels selected by the classifier comprises 6 channels.

4. The method of claim 3, wherein each of the 16 channels comprises an electrode affixed to a crown of the subject's head.

5. The method of claim 1, wherein each of the plurality of wavelength signals comprises a unique wavelength signal.

6. The method of claim 1, wherein the foot of the subject is segmented into eight distinct regions,
wherein each of the plurality of sensors on the foot of the subject is fixed to at least one of the eight distinct regions,
wherein each of the plurality of sensors on the foot of the subject cannot be fixed to the same distinct region.

7. The method of claim 1, wherein the subject's state is at least one of a sitting position, a standing position, and a walking movement.

8. A walking gait analysis apparatus comprising:
a computer controller;
a brain control interface (BCI);
and a plurality of sensors;
wherein the sensors are disposed on a toe, a midfoot, and a heel of an insole,
the plurality of sensors outputting a plurality of wavelength signals,
the plurality of wavelength signals corresponding to an applied plantar pressure from a foot of a subject on the insole,
the plurality of sensors connecting to an optical circulator, a light source, and an optical interrogator,
wherein the computer controller is configured to receive the plurality of wavelength signals,
wherein the computer controller is configured to receive, with processing circuitry, the plurality of wavelength signals corresponding to the applied plantar pressure from the foot of the subject and an electroencephalography (EEG) signal corresponding to the brain signals of the subject,
wherein the EEG signal is transmitted to the processing circuitry of the computer controller by the BCI mounted on the subject's head,
wherein the plurality of wavelength signals and the brain signals correspond to a state of the subject,
wherein the processing circuitry of the computer controller receives a plurality of wavelength signals corresponding to an applied plantar pressure from a foot of the subject,
the applied plantar pressure from the foot of the subject corresponding with the state of the subject,
wherein the processing circuitry of the computer controller receives a plurality of electroencephalography (EEG) signals corresponding to brain signals of the subject,
wherein each signal of the plurality of EEG signals corresponds with one signal of the plurality of wavelength signals, and wherein each signal of the plurality of EEG signals is registered by one channel of a plurality of channels on the BCI mounted on the subject's head,
wherein the plurality of channels transmits the plurality of EEG signals to a classifier,
wherein the processing circuitry of the computer controller trains the classifier using the plurality of EEG signals,
the classifier identifying a correlation between the plurality of EEG signals and the plurality of wavelength signals,
wherein the classifier selects a subsection of channels from the plurality of channels with a high correlation to the plurality of wavelength signals,
wherein the processing circuitry of the computer controller combines the subsection of channels with the plurality of wavelength signals to form a secondary dataset,
the secondary dataset being passed to a machine learning model,
the processing circuitry of the computer controller training the machine learning model using the secondary dataset to generate a walking gait analysis signal corresponding to a subject's state.

9. The walking gait apparatus of claim 8, wherein the state of the subject is at least one of a sitting position, standing position, or a walking movement.

10. The walking gait analysis apparatus of claim 8, wherein each of the plurality of sensors possess a baseline wavelength signal,
wherein a wavelength shift signal is calculated by the optical interrogator based on a difference between the baseline wavelength signal and a peak wavelength signal,
wherein each of the plurality of sensors produces the peak wavelength signal corresponding to the applied plantar pressure from the foot of the subject.

11. The walking gait analysis apparatus of claim 8, wherein the plurality of sensors comprises:
a first sensor,
the first sensor being fixed to the toe;
a second sensor,
the second sensor being fixed to the midfoot;
and a third sensor,
the third sensor being fixed to the heel.

12. The walking gait apparatus of claim 8, further comprising,
a wearable sandal arrangement, the insole, and a releasable strap-on,
wherein the insole is fixed to the underside of the wearable sandal arrangement,
wherein the releasable strap-on is fixed to the top of the wearable sandal arrangement.

13. The walking gait apparatus of claim 8, wherein the plurality of sensors is coated with a protective layer.

14. A system of plantar pressure response, wherein an applied plantar pressure is registered by a plurality of fiber bragg grating (FBG) sensors,
the plurality of FBG sensors being disposed on an insole,
wherein a light source illuminates the plurality of FBG sensors,
the plurality of FBG sensors each outputting a wavelength shift in response to an applied plantar pressure,
wherein an optical circulator provides a three-way gateway between the light source, an optical interrogator, and the plurality of FBG sensors,
the optical circulator being connected to the optical interrogator,
wherein the wavelength shift travels through the optical circulator from the plurality of FBG sensors to the optical interrogator,
wherein the optical interrogator displays the wavelength shift corresponding to each of the plurality of FGB sensors.

15. The system of claim 14, wherein each of the plurality of FGB sensors possess a base wavelength,
the base wavelength of each of the plurality of FGB sensors being a unique wavelength,
wherein the wavelength shifts of each of the plurality of FGB sensors do not overlap,
wherein a wavelength signal is calculated, via processing circuitry of the optical interrogator, as the difference between the base wavelength and the wavelength shift.

16. The system of claim 14, wherein each of the plurality of FGB sensors outputs a unique wavelength shift in response to an applied plantar pressure.

17. The system of claim 14, wherein the interrogation monitor includes a display unit,
wherein the wavelength shift from each of the plurality of FGB is projected on the display unit.

18. The system of claim 14, wherein a region ranging from 10 nanometers to 20 nanometers along a fiber length of the FBG is etched by ultraviolet (UV) radiation.
